SEQUENCE LISTING (28 sequences)

SEQ ID NO: 1 - DNA, 19 bp, Homo sapiens
tcgaaatgagaagaagagg

SEQ ID NO: 2 - DNA, 19 bp, Homo sapiens
cctcttcttctcatttcga

SEQ ID NO: 3 - DNA, 10 bp, Artificial Sequence
  synthetic peptide nucleic acid
tcttcttctc

SEQ ID NO: 4 - DNA, 10 bp, Homo sapiens
gagaagaaga

SEQ ID NO: 5 - DNA, 15 bp, Artificial Sequence
  synthetic peptide nucleic acid
tcttcttctcatttc

SEQ ID NO: 6 - DNA, 15 bp, Homo sapiens
gaaatgagaagaaga

SEQ ID NO: 7 - DNA, 15 bp, Artificial Sequence
  synthetic peptide nucleic acid
tcttcttctcatttc

SEQ ID NO: 8 - DNA, 15 bp, Homo sapiens
tcttcttctcatttc

SEQ ID NO: 9 - DNA, 15 bp, Homo sapiens
tcctcgagaagaaga

SEQ ID NO: 10 - DNA, 23 bp, Homo sapiens
ctgccgctgcttgtcatggtcat

SEQ ID NO: 11 - DNA, 23 bp, Artificial Sequence
  sequence introduced by donor 591
ctgccctaagctgtcatggtcat

SEQ ID NO: 12 - DNA, 23 bp, Artificial Sequence
  sequence introduced by donor 597
ctgccgctgctctgaggggtcat

SEQ ID NO: 13 - DNA, 60 bp, Artificial Sequence
  synthetic antisense oriented donor oligonucleotide targeting the CCR5 gene
attcccgagtagcagatgaccatgacagcttagggcaggaccagccccaagatgactatc

SEQ ID NO: 14 - DNA, 60 bp, Artificial Sequence
  synthetic antisense oriented donor oligonucleotide targeting the CCR5 gene
tttaggattcccgagtagcagatgacccctcagagcagcggcaggaccagccccaagatg

SEQ ID NO: 15 - DNA, 65 bp, Artificial Sequence
  synthetic donor oligonucleotide designed to span the detlta32 deletion site
gatgactatctttaatgtctggaaattcttccagaattaattaagactgtatggaaaatgagagc

SEQ ID NO: 16 - DNA, 66 bp, Artificial Sequence
  synthetic donor oligonucleotide designed to span the detlta32 deletion site
ccccaagatgactatctttaatgtctggaacgatcatcagaattgatactgactgtatggaaaatg

SEQ ID NO: 17 - DNA, 65 bp, Artificial Sequence
  synthetic donor oligonucleotide designed to span the detlta32 deletion site
gatgactatctttaatgtctggaaattctactagaattgatactgactgtatggaaaatgagagc

SEQ ID NO: 18 - DNA, 20 bp, Artificial Sequence
  synthetic allele-specific PCR forward primer “ASFUP”
gtcctgccgctgctctgagg

SEQ ID NO: 19 - DNA, 23 bp, Artificial Sequence
  synthetic allele-specific PCR reverse primer “ASR1”
gtgtaaactgagcttgctcgctc

SEQ ID NO: 20 - DNA, 24 bp, Artificial Sequence
  synthetic CCR5 gene-specific forward primer
acctttggggtggtgacaagtgtg

SEQ ID NO: 21 - DNA, 24 bp, Artificial Sequence
  synthetic CCR5 gene-specific reverse primer
tctccccgacaaaggcatagatga

SEQ ID NO: 22 - DNA, 22 bp, Artificial Sequence
  synthetic allele-specific PCR forward primer “JDCDELASF”
cattttccatacagtcttaatt

SEQ ID NO: 23 - DNA, 30 bp, Artificial Sequence
  synthetic allele-specific forward primer “JDCASF”
ccatacagtcagtatcaattctgatgatcg

SEQ ID NO: 24 - DNA, 32 bp, Artificial Sequence
  Restriction endonuclease selective PCR forward primer “RSB32PSPF”
gctctcattttccatacagtcagtatcaatcc

SEQ ID NO: 25 - DNA, 33 bp, Artificial Sequence
  Restriction endonuclease selective PCR reverse primer “CCR5RSBR2”
gtagggagcccagaagagaaaataaacaatcat

SEQ ID NO: 26 - DNA, 10 bp, Artificial Sequence
  synthetic peptide nucleic acid
tcttcttctc

SEQ ID NO: 27 - DNA, 15 bp, Artificial Sequence
  synthetic peptide nucleic acid
tcttcttctcatttc

SEQ ID NO: 28 - DNA, 15 bp, Artificial Sequence
  synthetic peptide nucleic acid
tcttcttctcatttc

(19) United States
(12) Patent Application Publication
(10) Pub. No.: US 2011/0262406 A1
(43) Pub. Date: Oct. 27, 2011

(54) COMPOSITIONS AND METHODS FOR TARGETED INACTIVATION OF HIV CELL SURFACE RECEPTORS

(75) Inventors: Jacob del Campo, North Haven, CT (US); Erica Beth Schleifman, New Haven, CT (US); Ranjit S. Bindra, New York, NY (US); Peter M. Glazer, Guilford, CT (US)

(73) Assignees: Yale University; Helix Therapeutics, Inc.

(21) Appl. No.: 13/091,918

(22) Filed: Apr. 21, 2011

Related U.S. Application Data
(60) Provisional application No. 61/326,551, filed on Apr. 21, 2010.

(51) Int. Cl. A61K 35/12; A61P 31/18; A61K 31/7088; C12N 15/01; C12N 5/071

(52) U.S. Cl. 424/93.7; 435/441; 435/366; 514/44 R

(57) ABSTRACT

Compositions for targeted mutagenesis of cell surface receptors for HIV and methods of their use are provided herein. The compositions include triplex-forming molecules that displace the polypyrimidine strand of target duplex and form a triple-stranded structure and hybrid duplex in a sequence specific manner with the polypurine strand of the target duplex. The triplex-forming molecules include a mixed-sequence “tail” which increases the stringency of binding to the target duplex, improves the frequency of modification at the target site, and reduces the requirement for a polypurine:polypyrimidine stretch. Methods for using the triplex-forming molecules in combination with one or more donor oligonucleotides for targeted modification of sites within or adjacent to genes that encodes cell surface receptors for human immunodeficiency virus (HIV) are also disclosed. Methods for ex vivo and in vivo prophylaxis and therapy of HIV infection using the disclosed compositions are also provided.

### CROSS-REFERENCE TO RELATED APPLICATIONS

This application claims benefit of and priority to U.S. Ser. No. 61/326,551, filed Apr. 21, 2010, which is incorporated by reference in its entirety.

### STATEMENT REGARDING FEDERALLY SPONSORED RESEARCH OR DEVELOPMENT

This invention was made with Government Support under Agreement Nos. R01CA064186 and R01HL082655 awarded to Peter M. Glazer by the National Institutes of Health. The Government has certain rights in the invention.

### FIELD OF THE INVENTION

The present disclosure generally relates to the field of compositions that bind to DNA encoding cell surface receptors for HIV and methods of using these compositions.

### REFERENCE TO SEQUENCE LISTING

The Sequence Listing being submitted herewith as a text file named “HT_{—}101 YU 5346_ST25.txt,” created on Apr. 20, 2011, and having a size of 7,818 bytes is hereby incorporated by reference pursuant to 37 C.F.R. §1.52(e)(5).

### BACKGROUND OF THE INVENTION

HIV-1 is a member of the Retroviridae family belonging to the genus lentiviruses. The Retroviridae are enveloped viruses containing two positive sense RNA strands that are converted into dsDNA by the highly error-prone viral reverse transcriptase enzyme generating isolate diversity by both point mutation and intergenomic recombination. HIV-1 isolates fall into three groups: M (Major/Main), N (Non-M, Non-O/New) and O (Outlier) of which, as implied, group M is most common. Group M is subdivided into several subtypes or clades (A-D, F-H, J and K), of which B is most common in the Western world, while C is the predominant subtype found primarily in India, China and sub-Saharan Africa. The remaining subtypes, as well as HIV-1 variants with characteristics of several different subtypes, so-called circulating recombinant forms (CRFs), are dispersed throughout Africa and other parts of the world.

HIV-1 contains the exterior envelope glycoprotein, gp120, and the transmembrane glycoprotein, gp41. These proteins are generated by cleavage of a heavily glycosylated precursor protein, gp160, by furin-like enzymes during transport through the Golgi apparatus. Once transported to the cell surface, trimeric gp120/gp41 envelope glycoprotein spikes are incorporated into budding virus for release of new HIV-1 particles. Each new infectious cycle is initiated when the external envelope glycoprotein gp120 binds the primary receptor, CD4, which is embedded in the plasma membrane on the surface of potential target cells. Interaction of gp120 with CD4 is followed by a series of conformational changes in Env resulting in exposure of a transient binding site that allows the spike to interact with its co-receptor, usually CCR5 or CXCR4. This in turn promotes additional conformational changes that allow gp41 to insert its fusion peptide into the target cell membrane to form a prehairpin structure, which then collapses into an energetically stable six-helix bundle structure, driving virus-to-cell membrane fusion and entry of the HIV-1 core into the target cell. This sequence of events occurs at the plasma membrane at neutral pH.

Entry inhibitors have recently emerged as a new class of HIV therapeutics which could potentially change the treatment paradigm. These drugs block cell surface receptors required for HIV entry into T-cells. R5-tropic HIV infection requires cell surface expression of both the primary receptor, CD4, and CCR5, a seven-transmembrane G protein-coupled co-receptor, for viral entry. Hence, the CCR5 co-receptor has become an attractive target for the development of small-molecule entry inhibitors as well as for gene therapy (Tsibris and Kuritzkes, Annu Rev Med, 58:445-59 (2007), Perez et al., Nat. Biotechnol., 26:808-16 (2008)). A naturally occurring 32-bp deletion (CCR5-delta32) results in a truncated protein that fails to localize to the cell surface (Liu et al., Cell, 86(3):367-77 (1996)), providing individuals homozygous for this mutation with immunity to R5 tropic HIV-1 (Samson et al., Nature 382(6593):722-5 (1996))). In a case report published in the New England Journal of Medicine an HIV-1 positive patient with acute myeloid leukemia received an allogeneic stem cell transplant with cells that were homozygous for the CCR5-delta32 mutation (Hatter et al., N Engl J Med, 360:692-8 (2009)). Post-transplantation the recipient had no detectable virus and had increased CD4 counts, demonstrating reconstitution of the immune system. Individuals who possess a homozygous inactivating mutation (referred to as the Delta32 mutation, or Δ32 mutation) in the CCR5 gene are almost completely resistant to infection by R5-tropic HIV-1 strains, with no other significant adverse consequences.

With over 40 million people currently living with AIDS, industry analysts estimate that a successful therapy targeting CCR5 will generate sales of $500-700 mM per year. A number of pharmaceutical companies are currently trying to develop entry-inhibitor drugs to block the receptor protein, although progress has been hindered by toxicity, efficacy and drug resistance.

Since the initial observation of triple-stranded DNA many years ago by Felsenfeld et al., J. Am. Chem. Soc. 79:2023 (1957), oligonucleotide-directed triple helix formation has emerged as a valuable tool in molecular biology. Current knowledge suggests that oligonucleotides can bind as third strands of DNA in a sequence specific manner in the major groove in polypurine/polypyrimidine stretches in duplex DNA. In one motif, a polypyrimidine oligonucleotide binds in a direction parallel to the purine strand in the duplex, as described by Moser and Dervan, Science 238:645 (1987), Praseuth et al., Proc. Natl. Acad. Sci. USA 85:1349 (1988), and Mergny et al., Biochemistry 30:9791 (1991). In the alternate purine motif, a polypurine strand binds anti-parallel to the purine strand, as described by Beal and Dervan, Science 251:1360 (1991). The specificity of triplex formation arises from base triplets (AAT and GGC in the purine motif) formed by hydrogen bonding; mismatches destabilize the triple helix, as described by Mergny et al., Biochemistry 30:9791 (1991) and Beal and Dervan, Nuc. Acids Res. 11:2773 (1992).

Triplex forming oligonucleotides (TFOs) are useful for several molecular biology techniques. For example, triplex forming oligonucleotides designed to bind to sites in gene promoters have been used to block DNA binding proteins and to block transcription both in vitro and in vivo. (Maher et al., Science 245:725 (1989), Orson et al., Nucleic Acids Res. 19:3435 (1991), Postal et al., Proc. Natl. Acad. Sci. USA 88:8227 (1991), Cooney et al., Science 241:456 (1988), Young et al., Proc. Natl. Acad. Sci. USA 88:10023 (1991), Maher et al., Biochemistry 31:70 (1992), Duval-Valentin et al., Proc. Natl. Acad. Sci. USA 89:504 (1992), Blume et al., Nucleic Acids Res. 20:1777 (1992), Durland et al., Biochemistry 30:9246 (1991), Grigoriev et al., J. of Biological Chem. 267:3389 (1992), and Takasugi et al., Proc. Natl. Acad. Sci. USA 88:5602 (1991)). Site specific cleavage of DNA has been achieved by using triplex forming oligonucleotides linked to reactive moieties such as EDTA-Fe(II) or by using triplex forming oligonucleotides in conjunction with DNA modifying enzymes (Perrouault et al., Nature 344:358 (1990), Francois et al., Proc. Natl. Acad. Sci. USA 86:9702 (1989), Lin et al., Biochemistry 28:1054 (1989), Pei et al., Proc. Natl. Acad. Sci. USA 87:9858 (1990), Strobel et al., Science 254:1639 (1991), and Posvic and Dervan, J. Am. Chem. Soc. 112:9428 (1992)). Sequence specific DNA purification using triplex affinity capture has also been demonstrated. (Ito et al., Proc. Natl. Acad. Sci. USA 89:495 (1992)). Triplex forming oligonucleotides linked to intercalating agents such as acridine, or to cross-linking agents, such as p-azidophenacyl and psoralen, have been utilized, to enhance the stability of triplex binding. (Praseuth et al., Proc. Natl. Acad. Sci. USA 85:1349 (1988), Grigoriev et al., J. of Biological Chem. 267:3389 (1992), Takasugi et al., Proc. Natl. Acad. Sci. USA 88:5602 (1991)).

Gene therapy can be defined by the methods used to introduce heterologous DNA into a host cell or by the methods used to alter the expression of endogenous genes within a cell. As such, gene therapy methods can be used to alter the phenotype and/or genotype of a cell.

Targeted modification of the genome by gene replacement is of value as a research tool and in gene therapy. However, while facile methods exist to introduce new genes into mammalian cells, the frequency of homologous integration is limited (Hanson et al., Mol. Cell. Biol. 15(1), 45-51 (1995), and isolation of cells with site-specific gene insertion typically requires a selection procedure (Capecchi, M. R., (1989) Science 244(4910), 1288-1292). Site-specific DNA damage in the form of double-strand breaks produced by rare cutting endonucleases can promote homologous recombination at chromosomal loci in several cell systems, but this approach requires the prior insertion of the recognition sequence into the locus.

Methods which alter the genotype of a cell typically rely on the introduction into the cell of an entire replacement copy of a defective gene, a heterologous gene, or a small nucleic acid molecule such as an oligonucleotide, to treat human, animal and plant genetic disorders. The introduced gene or nucleic acid molecule, via genetic recombination, replaces the endogenous gene. This approach requires complex delivery systems to introduce the replacement gene into the cell, such as genetically engineered viruses, or viral vectors.

Alternatively, gene therapy methods can be used to alter the expression of an endogenous gene. One example of this type of method is antisense therapy. In antisense therapy, a nucleic acid molecule is introduced into a cell, the nucleic acid molecule being of a specific nucleic acid sequence so as to hybridize or bind to the mRNA encoding a specific protein. The binding of the antisense molecule to an mRNA species decreases the efficiency and rate of translation of the mRNA.

Gene therapy is being used on an experimental basis to treat well known genetic disorders of humans such as retinoblastoma, cystic fibrosis, and globinopathies such as sickle cell anemia. However, in vivo efficiency is low due to the limited number of recombination events actually resulting in replacement of the defective gene.

Strategies for modifying immune cells to resist HIV-1 include CCR5-directed ribozymes or siRNAs directed against HIV-1-encoded RNAs, but these require continual, long-term expression for activity (Akkina et al., Anticancer Res., 23(3A):1997-2005 (2003), Amado et al., Hum Gene Ther, 15(3):251-62 (2004), Anderson, R. Akkina, Retrovirology, 2:53 (2005). The use of zinc-finger nucleases to induce mutagenic double-strand breaks in the CCR5 gene in CD4⁺ T-cells has been reported (Perez et al., Nat. Biotechnol., 26:808-16 (2008)). However, expression of zinc-finger nucleases requires complex viral vectors to introduce large plasmid constructs, and the safety profile of such nucleases is unknown. In addition, nuclease-induced double-strand breaks lead to an unpredictable mixture of mutations. Alternatively, the triplex-forming molecule approaches described herein typically utilize chemically synthesized oligonucleotides, a class of agents that has been extensively studied in clinical trials with favorable safety profile (Vasquez, et al., Nucleic Acids Res., 27:1176-81 (1999); Vasquez, et al., Science, 290:530-3 (2000)).

Methods for targeted gene therapy using triplex-forming oligonucleotides (TFO's) and peptide nucleic acids (PNAs) are described in U.S. Application No. 20070219122 and their use for treating infectious diseases such as HIV are described in U.S. Application No. 2008050920. However, as described below, these compositions require a lengthy polypurine:polypyrimidine target sequence, limiting the number of potential targets. Furthermore, previous attempts to target CCR5 using chlorambucil-conjugated DNA-based TFO's was accomplished only in detergent permeabilized and therefore dead cells.

Compositions and methods for targeted mutagenesis of genes encoding cell surface receptors for HIV in living cells would be useful as a means of gene therapy for ex vivo and in vivo prophylactic and therapeutic applications. Such compositions and methods would also be useful for generating cells with a spectrum of mutations in genes encoding cell surface receptors for HIV.

Therefore it is an object of the invention to provide triplex-forming molecules having improved flexibility in the design of the target sequence; and/or greater stringency of binding to the target sequence than TFOs or bis-PNAs; and/or a higher frequency of recombination of a donor oligonucleotide sequence compared to TFOs or bis-PNAs targeting the same duplex DNA sequence in living cells.

It is a further object of the invention to provide methods of use thereof for in vivo and ex vivo targeted recombination at sites of or adjacent to genes that encode cell surface receptors for HIV.

It is a still further object of the invention to provide cells that contain mutations at sites of or adjacent to genes that encode cell surface receptors for HIV.

It is a further object of the present invention to provide compositions and methods for treating or preventing HIV infection by gene therapy without the need for a viral vector.

It is a further object of the invention to provide compositions and methods for treating or preventing HIV infection by ex vivo or in vivo gene therapy.

### SUMMARY OF THE INVENTION

Compositions for targeted mutagenesis of cell surface receptors for HIV and methods of their use are provided herein. The compositions include triplex-forming molecules that displace the polypyrimidine strand of target duplex and form a triple-stranded structure and hybrid duplex in a sequence specific manner with the polypurine strand of the target duplex. Triplex-forming molecules can include a pair of molecules, or a pair of molecules connected by a linker, that facilitate strand displacement and triplex formation, referred to as a “clamp,” in which one molecule binds to the target strand by Hoogsteen binding and the other molecule binds to the target strand by Watson-Crick binding in a sequence specific manner. The triplex-forming molecules preferably include a Watson-Crick binding “tail” added to the end of the Watson-Crick binding portion of the clamp. The tail includes additional nucleobases that bind to the target strand outside the triple helix formed at the site of duplex stand displacement, and hybridize as a duplex with the polypurine strand of the target duplex. The tail increases the stringency of binding to the target duplex, improves the frequency of modification at the target site and reduces the requirement for a polypurine:polypyrimidine stretch compared to triplex forming oligonucleotide (TFOs) or peptide nucleic acids (PNAs), thereby increasing the number of potential binding sites. Triplex-forming molecules may be composed of peptide nucleic acids, or a suitable substitute oligonucleotide with a backbone having low negative charge, no charge or positive charge to facilitate clamp formation at the target site.

The target site is within or adjacent to a gene that encodes a cell surface receptor for human immunodeficiency virus (HIV). The HIV cell surface receptor can be a chemokine receptor, including CXCR4, CCR5, CCR2b, CCR3 and CCR1. The target site can be within the coding region of the gene. The target sequence is preferably within or adjacent to a portion of the HIV cell surface receptor gene important to its function in allowing HIV entry into cells, such as nucleotides or nucleotide sequences involved in efficient expression of the receptor, transport of the receptor to the cell surface, stability of the receptor, viral binding by the receptor, or endocytosis of the receptor. In one embodiment, the target site for the triplex-forming molecule is within or adjacent to the human CCR5 gene. In a preferred embodiment, the target site encompasses or is adjacent to the site of a naturally occurring nonsense mutation referred to as the Δ32 mutation.

The triplex-forming molecules can also be tail clamp peptide nucleic acids (tcPNAs). Highly stable PNA:DNA:PNA triplex structures can be formed from strand invasion of a duplex DNA with two PNA strands. In this complex, the PNA/DNA/PNA triple helix portion and the PNA/DNA duplex portion both produce displacement of the pyrimidine-rich triple helix, creating an altered structure that has been shown to strongly provoke the nucleotide excision repair pathway and to activate the site for recombination with the donor oligonucleotide. The two PNA strands can be linked together to form a bis-PNA molecule.

The triplex-forming molecules are useful to induce site-specific homologous recombination in mammalian cells when used in combination with one or more donor oligonucleotides. Donor oligonucleotides can be tethered to triplex-forming molecules or can be separate from the triplex-forming molecules. The donor oligonucleotides can contain at least one nucleotide mutation, insertion or deletion relative to the target duplex DNA. Triplex-forming molecules can be used in conjunction with donor oligonucleotides to cause mutations in HIV cell surface receptor genes that result in one or more deficiencies in the ability of the encoded receptor to bind to HIV and allow its transport into the cell. Suitable mutations are those that result in a decrease in the expression of a cell surface HIV receptor, its transport to the cell surface, its stability, its ability to bind to HIV, or its endocytosis.

Also provided are cell lines generated by contacting cells with triplex-forming molecules and donor oligonucleotides that contain at least one mutation in a cell surface receptor for HIV. The cells are preferably hematopoietic in origin. Useful hematopoietic cells include T cells and hematopoietic stem cells including CD34⁺ cells. The cell lines can be used to identify new targets that confer resistance to HIV, and for the screening and development of other HIV therapeutic agents, including other agents that reduce or inhibit the entry of HIV into cells, or in therapy.

Also provided are prophylactic and therapeutic methods for treating subjects with or at risk of developing an HIV infection using the compositions disclosed herein. The methods can be used to prevent infection of an individual with HIV or to reduce the viral load of an individual already infected with HIV. In one embodiment, ex vivo therapy is used for treatment or prevention of HIV infection. These methods include isolating target cells, contacting the target cells ex vivo with triplex-forming molecules and donor oligonucleotides to cause targeted mutagenesis of HIV cell surface receptor genes, expanding the modified cells in culture, and administering the modified cells to the subject in need thereof. The cells can be isolated from the subject to be treated or can be isolated from a syngenic or allogenic host. The cells can be hematopoietic stem cells and are preferably CD34⁺ cells. In another embodiment, the modified cells are differentiated in ex vivo culture and expanded in large numbers prior to administration to the subject. The cells are preferably differentiated into CD4⁺ T cells. Methods for treating or preventing HIV infection by administering the compositions disclosed herein are also provided.

### BRIEF DESCRIPTION OF THE DRAWINGS

FIGS. 1A, 1B and 1C are diagrams depicting the binding of PNA or tcPNA to a subsequence of 5′ TCGAAATGAGAAGAAGAGG 3′ (SEQ ID NO: 1), a polypurine target site in CCR5. The complimentary sequence of SEQ ID NO: 1 is 5′ CCTCTTCTTCTCATTTCGA 3′ (SEQ ID NO: 2). FIG. 1A shows the sequence JTJTTJTTJT-OOO-TCTTCTTCTC (SEQ ID NO: 26) from PNA-679 binding to subsequence 5′ GAGAAGAAGA 3′ (SEQ ID NO: 4). FIG. 1B shows the sequence JTJTTJTTJT-OOO-TCTTCTTCTCATTTC (SEQ ID NO: 27) from tcPNA-679 binding to subsequence 5′ GAAATGAGAAGAAGA 3′ (SEQ ID NO: 6). FIG. 1C shows the sequence JTTJT-OOO-TCTTCTTCTCATTTC (SEQ ID NO: 28) from tcPNA-684 binding to subsequence 5′ GAAATGAGAAGAAGA 3′ (SEQ ID NO: 6). PNA sequences are from N-terminus to C-terminus; J=pseudoisocytosine, O=flexible 8-amino-3,6-dioxaoctanoic acid, 6-aminohexanoic acid monomers; and both ends of each PNA are capped with three lysines (Lys).

FIG. 2 is a diagram depicting PNA-stimulated modification of the CCR5 gene (gray rectangle) in human THP-1 cells. The positions of the 32 base pair deletion (Δ 32 mutation, position 554-585) the resulting stop codon (X) it creates at position 678, are relative to the ATG start codon at position 1. The insert between positions 674 and 688 show the polypurine:polypyrimidine stretch of genomic DNA from 5′ TCTTCTTCTCATTTC 3′ (SEQ ID NO: 8) and its complementary sequence 5′ GAAATGAGAAGAAGA 3′ (SEQ ID NO: 9) where the polypurine target sequence of the PNA binding site is bolded.

FIGS. 3A-F are diagrams depicting the binding of PNAs described above in FIG. 1, binding to wildtype and mutant CCR5 sequence. FIG. 3A shows tcPNA-684: Lys-Lys-Lys-JTTJT-OOO-TCTTCTTCTCATTTC-Lys-Lys-Lys (SEQ ID NO: 7) binding to wildtype CCR5 sequence 5′ GAAATGAGAAGAAGA 3′ (SEQ ID NO: 6). FIG. 3B shows tcPNA-684 (SEQ ID NO: 7) binding to mutant CCR5 sequence 5′ TCCTCGAGAAGAAGA 3′ (SEQ ID NO: 9). FIG. 3C shows bis PNA-679: Lys-Lys-Lys-JTJTTJTTJT-OOO-TCTTCTTCTC-Lys-Lys-Lys (SEQ ID NO: 3) binding to wildtype CCR5 sequence 5′ GAGAAGAAGA 3′ (SEQ ID NO: 4). FIG. 3D shows tcPNA-679: Lys-Lys-Lys-JTJTTJTTJT-OOO-TCTTCTTCTCATTTC-Lys-Lys-Lys (SEQ ID NO: 5) binding to wildtype sequence 5′ GAAATGAGAAGAAGA 3′ (SEQ ID NO: 6). FIG. 3E shows tcPNA-679 (SEQ ID NO: 5) binding to mutant CCR5 sequence 5′ TCCTCGAGAAGAAGA 3′ (SEQ ID NO: 9). FIG. 3F shows bis PNA-679 (SEQ ID NO: 3) binding to wildtype CCR5 sequence 5′ GAGAAGAAGA 3′ (SEQ ID NO: 4).

FIG. 4 is a schematic of PNA-induced recombination between the CCR5 gene and two different antisense oriented 60-nucleotide donor oligonucleotides, donor 591 and donor 597. The donor oligos were designed to be homologous to a portion of the CCR5 gene (5′ CTGCCGCTGCTTGTCATGGTCAT 3′ (SEQ ID NO: 10)) except for a central 6-bp (shown capitalized and bolded) segment intended to introduce, via recombination, an in-frame stop codon (shown underlined). The sequence introduced by each donor is compared to wildtype (SEQ ID NO: 10). Donor 591:5′ CTGCCCTAAGCTGTCATGGTCAT 3′ (SEQ ID NO: 11) and 5′ CTGCCGCTGCTCTGAGGGGTCAT 3′ (SEQ ID NO: 12). The relative position of the tcPNA-679 binding site is also shown.

FIG. 5 is a schematic of the CCR5 gene showing the relative positions of the antisense oriented donors 591 and 597, the binding site of tcPNA-679, and Wildtype (WT)/Allele-specific forward “F” primer and Wildtype (WT)-specific reverse “R” primer used for allele specific PCR (AS-PCR). The forward primer contains either the 6-base pair mutation or the wildtype sequence.

FIG. 6 is a bar graph showing the relative CCR5 mRNA abundance for untreated (open bars) and PMA treated (closed bars) THP-1 as determined by a Taqman real-time PCR assay.

FIG. 7 is histogram showing CCR5 protein cell surface expression (% of Max) as determined by antibody staining and flow cytometry (FACS) detection of PE Fluorescence (FL-2), for experimental (solid line) and control (hashed-line).

FIG. 8 is a bar graph illustrating the results of FACS analysis for CCR5 protein expression in THP-1 cells following targeted modification of the CCR5 gene. The graph shows the percentage of cells positive for CCR5 protein expression on (bars read from left to right) wildtype (WT THP-1) cells, cells mutant for the 597 mutation (Mut597/WT clone), or cells mutant for the 597 mutation and 591 mutation (Mut597/Mut591 clone).

FIG. 9 is a bar graph showing the p24 concentration (picograms/milliliter) in wildtype (WT THP-1; clear bars) and cells heterozygous for the 591 mutation (Mut591/WT; black bars) as a function of days (4, 6, 8, 10, or 12) after viral challenge with R5-tropic HIV-1 infection. The graph shows the mean and standard deviation (error bars) of duplicate determinations from two wells.

FIG. 10 is a bar graph showing the relative mutation rate (value normalized to a gene specific product (GSP)) as measured by allele-specific PCT of genomic DNA isolated from CD34⁺ hematopoetic stem cells mock transfected, or transfected with either donor 597 alone, donor 597 and tcPNA-679 (from left to right as labeled). Mean results from two replicates are shown, with error bars indicating standard deviation.

FIG. 11 is a bar graph showing the targeted gene modification frequency (targeting frequency (%)) for 597 donor oligo alone (left hand bar) or in combination with PNA-679 (right hand bar).

FIG. 12 is a schematic showing the mouse model utilized to investigate engraftment of human CD34+ hematopoetic stems carrying a modified CCR5.

FIG. 13 is a bar graph showing the relative mutation rate (value normalized to a gene specific product (GSP)) as measured by allele-specific PCT with donor 597-specific primers of genomic DNA isolated from spleen of mice that were injected with non-humanized CD 34+ cells, untreated human CD 34+ cells, or human CD 34+ cells transfected with tc-PNA-679 plus donor 597 and donor 591, (from left to right as labeled).

FIG. 14 is a bar graph showing the relative mutation rate (value normalized to a gene specific product (GSP)) as measured by allele-specific PCT with donor 591-specific primers of genomic DNA isolated from spleen of mice that were injected with non-humanized CD 34+ cells, untreated human CD 34+ cells, or human CD 34+ cells transfected with tc-PNA-679 plus donor 597 and donor 591, (from left to right as labeled).

FIG. 15 is a bar graph showing the relative mutation rate (value normalized to a gene specific product (GSP)) as measured by allele-specific PCT with donor 597-specific primers (left bar within each pair) or donor 591-specific primers (right bar with each pair) of genomic DNA isolated from CD4+ cells isolated from control (left-hand pair of bars), and transplanted (right-hand pair of bars) mice.

### DETAILED DESCRIPTION OF THE INVENTION

### I. Compositions that Bind to Double-Stranded DNA Encoding Cell Surface Receptors for HIV

A. Triplex-Forming Molecules

Disclosed herein are compositions containing molecules, referred to as triplex-forming molecules, which bind to duplex DNA in a sequence-specific manner to form a triple-stranded structure. The triplex-forming molecules can be used to induce site-specific homologous recombination in mammalian cells when combined with donor DNA molecules. The donor DNA molecules can contain mutated nucleic acids relative to the target DNA sequence. This is useful to activate, inactivate, or otherwise alter the function of a polypeptide or protein encoded by the targeted duplex DNA. As used herein “triplex-forming molecules” refer to a pair of single-stranded molecules, or a single molecule composed of a pair of molecules connected by a linker, that facilitate strand displacement and triplex formation, in which one molecule binds to the target strand by Hoogsteen binding and the other molecule binds to the target strand by Watson-Crick binding in a sequence specific manner (also referred to as a “clamp”). As used herein, a pair of single-stranded molecules, or a single molecule composed of a pair of molecules connected by a linker may be identified according to the Watson-Crick binding portion, and the Hoogsteen binding portion. As described below, the triplex-forming molecules can also have a Watson-Crick binding “tail” added to the end of the Watson-Crick binding portion of the clamp. The “tail” includes additional nucleobases that bind to the target strand outside the triple helix formed at the site of duplex strand displacement. For example, suitable triplex-forming molecules include, but are not limited to a pair of peptide nucleic acids (PNAs), or bis-PNAs. In one preferred embodiment, the triplex-forming molecule is a bis-PNA including a Watson-Crick binding PNA with a tail, and Hoogsteen binding PNA which is connected to the Wastson-Crick binding PNA by an O-linker.

1. Peptide Nucleic Acids

In one embodiment, the triplex-forming molecules are peptide nucleic acids (PNAs). Peptide nucleic acids are molecules in which the phosphate backbone of oligonucleotides is replaced in its entirety by repeating N-(2-aminoethyl)-glycine units and phosphodiester bonds are replaced by peptide bonds. The various heterocyclic bases are linked to the backbone by methylene carbonyl bonds. PNAs maintain spacing of heterocyclic bases that is similar to oligonucleotides, but are achiral and neutrally charged molecules. Peptide nucleic acids are comprised of peptide nucleic acid monomers. The heterocyclic bases can be any of the standard bases (uracil, thymine, cytosine, adenine and guanine) or any of the modified heterocyclic bases described below.

PNAs can bind to DNA via Watson-Crick hydrogen bonds, but with binding affinities significantly higher than those of a corresponding nucleotide composed of DNA or RNA. The neutral backbone of PNAs decreases electrostatic repulsion between the PNA and target DNA phosphates. Under in vitro or in vivo conditions that promote opening of the duplex DNA, PNAs can mediate strand invasion of duplex DNA resulting in displacement of one DNA strand to form a D-loop.

Highly stable triplex PNA:DNA:PNA structures can be formed from a homopurine DNA strand and two PNA strands. The two PNA strands may be two separate PNA molecules, or two PNA molecules linked together by a linker of sufficient flexibility to form a bis-PNA. In both cases, the PNA molecule(s) forms a triplex “clamp” with one of the strands of the target duplex while displacing the other strand of the duplex target. In this structure, one strand forms Watson-Crick base pairs with the DNA strand in the anti-parallel orientation (the Watson-Crick binding portion), whereas the other strand forms Hoogsteen base pairs to the DNA strand in the parallel orientation (the Hoogsteen binding portion). A homopurine strand allows formation of a stable PNA/DNA/PNA triplex. PNA clamps can form at shorter homopurine sequences than those required by triplex-forming oligonucleotides (TFOs) and also do so with greater stability.

Suitable molecules for use in linkers of bis-PNA molecules include, but are not limited to, 8-amino-3,6-dioxaoctanoic acid, referred to as an O-linker, and 6-aminohexanoic acid. Poly(ethylene) glycol monomers can also be used in bis-PNA linkers. A bis-PNA linker can contain multiple linker molecule monomers in any combination.

PNAs can also include other positively charged moieties to increase the solubility of the PNA and increase the affinity of the PNA for duplex DNA. Commonly used positively charged moieties include the amino acids lysine and arginine, although other positively charged moieties may also be useful. Lysine and arginine residues can be added to a bis-PNA linker or can be added to the carboxy or the N-terminus of a PNA strand.

### Chemical Modifications

In alternative embodiments, the triplex-forming molecules including PNAs and other suitable oligonucleotides may include one or more modifications or substitutions to the nucleobases or linkages. Modifications should not prevent, and preferably enhance, duplex invasion, strand displacement, and/or stabilize triplex formation as described above by increasing specificity or binding affinity of the triplex-forming molecules to the target site. Modified bases and base analogues, modified sugars and sugar analogues and/or various suitable linkages known in the art are also suitable for use in triplex-forming molecules such as PNAs.

a. Heterocyclic Bases

The principal naturally-occurring nucleotides comprise uracil, thymine, cytosine, adenine and guanine as the heterocyclic bases. Triplex-forming molecules such as PNAs can include chemical modifications to their nucleotide constituents. For example, target sequences with adjacent cytosines can be problematic. Triplex stability is greatly compromised by runs of cytosines, thought to be due to repulsion between the positive charge resulting from the N³ protonation or perhaps because of competition for protons by the adjacent cytosines. Chemical modification of nucleotides comprising triplex-forming molecules such as PNAs may be useful to increase binding affinity of triplex-forming molecules and/or triplex stability under physiologic conditions.

Chemical modifications of heterocyclic bases or heterocyclic base analogs may be effective to increase the binding affinity of a nucleotide or its stability in a triplex. Chemically-modified heterocyclic bases include, but are not limited to, inosine, 5-(1-propynyl) uracil (pU), 5-(1-propynyl) cytosine (pC), 5-methylcytosine, 8-oxo-adenine, pseudocytosine, pseudoisocytosine, 5 and 2-amino-5-(2′-deoxy-β-D-ribofuranosyl)pyridine (2-aminopyridine), and various pyrrolo- and pyrazolopyrimidine derivatives. Substitution of 5-methylcytosine or pseudoisocytosine for cytosine in triplex-forming molecules such as PNAs helps to stabilize triplex formation at neutral and/or physiological pH, especially in triplex-forming molecules with isolated cytosines. This is because the positive charge partially reduces the negative charge repulsion between the triplex-forming molecules and the target duplex, and allows for Hoogsteen binding.

b. Backbone

The nucleotide subunits of the triplex-forming molecules such as PNAs are connected by an internucleotide bond that refers to a chemical linkage between two nucleoside moieties. Peptide nucleic acids (PNAs) are synthetic DNA mimics in which the phosphate backbone of the oligonucleotide is replaced in its entirety by repeating N-(2-aminoethyl)-glycine units and phosphodiester bonds are typically replaced by peptide bonds. The various heterocyclic bases are linked to the backbone by methylene carbonyl bonds, which allow them to form PNA-DNA or PNA-RNA duplexes via Watson-Crick base pairing with high affinity and sequence-specificity. PNAs maintain spacing of heterocyclic bases that is similar to conventional DNA oligonucleotides, but are achiral and neutrally charged molecules. Peptide nucleic acids are comprised of peptide nucleic acid monomers.

Other backbone modifications, particularly those relating to PNAs, include peptide and amino acid variations and modifications. Thus, the backbone constituents of PNAs may be peptide linkages, or alternatively, they may be non-peptide linkages. Examples include acetyl caps, amino spacers such as 8-amino-3,6-dioxaoctanoic acid (referred to herein as O-linkers), amino acids such as lysine are particularly useful if positive charges are desired in the PNA, and the like. Methods for the chemical assembly of PNAs are well known. See, for example, U.S. Pat. Nos. 5,539,082, 5,527,675, 5,623,049, 5,714,331, 5,736,336, 5,773,571 and 5,786,571.

Backbone modifications used to generate triplex-forming molecules should not prevent the molecules from binding with high specificity to the target site and creating a triplex with the target duplex nucleic acid by displacing one strand of the target duplex and forming a clamp around the other strand of the target duplex.

2. Modified Nucleic Acids

Modified nucleic acids in addition to peptide nucleic acids are also useful as triplex-forming molecules. Oligonucleotides comprise a chain of nucleotides which are linked to one another. Each nucleotide typically comprises a heterocyclic base (nucleic acid base), a sugar moiety attached to the heterocyclic base, and a phosphate moiety which esterifies a hydroxyl function of the sugar moiety. The principal naturally-occurring nucleotides comprise uracil, thymine, cytosine, adenine and guanine as the heterocyclic bases, and ribose or deoxyribose sugar linked by phosphodiester bonds. As used herein “modified nucleotide” or “chemically modified nucleotide” defines a nucleotide that has a chemical modification of one or more of the heterocyclic base, sugar moiety or phosphate moiety constituents. Preferably the charge of the modified nucleotide is reduced compared to DNA or RNA oligonucleotides of the same nucleobase sequence. Most preferably the triplex-forming molecules have low negative charge, no charge, or positive charge such that electrostatic repulsion with the nucleotide duplex at the target site is reduced compared to DNA or RNA oligonucleotides with the corresponding nucleobase sequence.

Examples of modified nucleotides with reduced charge include modified internucleotide linkages such as phosphate analogs having achiral and uncharged intersubunit linkages (e.g., Sterchak, E. P. et al., Organic Chem., 52:4202, (1987)), and uncharged morpholino-based polymers having achiral intersubunit linkages (see, e.g., U.S. Pat. No. 5,034,506). Some internucleotide linkage analogs include morpholidate, acetal, and polyamide-linked heterocycles. Locked nucleic acids (LNA) are modified RNA nucleotides (see, for example, Braasch, et al., Chem. Biol., 8(1):1-7 (2001)). LNAs form hybrids with DNA which are more stable than DNA/DNA hybrids, a property similar to that of peptide nucleic acid (PNA)/DNA hybrids. Therefore, LNA can be used just as PNA molecules would be. LNA binding efficiency can be increased in some embodiments by adding positive charges to it. Commercial nucleic acid synthesizers and standard phosphoramidite chemistry are used to make LNAs.

Triplex-forming molecules may also include nucleotides with modified heterocyclic bases, sugar moieties or sugar moiety analogs. Modified nucleotides may include modified heterocyclic bases or base analogs as described above with respect to peptide nucleic acids. Sugar moiety modifications include, but are not limited to, 2′-O-aminoethoxy, 2′-O-amonioethyl (2′-OAE), 2′-O-methoxy, 2′-O-methyl, 2-guanidoethyl (2′-OGE), 2′-O,4′-C-methylene (LNA), 2′-O-(methoxyethyl) (2′-OME) and 2′-O-(N-(methyl)acetamido) (2′-OMA). 2′-O-aminoethyl sugar moiety substitutions are especially preferred because they are protonated at neutral pH and thus suppress the charge repulsion between the triplex-forming molecule and the target duplex. This modification stabilizes the C3′-endo conformation of the ribose or deoxyribose and also forms a bridge with the i-1 phosphate in the purine strand of the duplex.

Triplex-forming molecules such as PNAs may optionally include one or more terminal amino acids at either or both termini to increase stability, and/or affinity of the PNAs or modified nucleotides for DNA, or increase solubility of PNAs or modified nucleotides. Commonly used positively charged moieties include the amino acids lysine and arginine, although other positively charged moieties may also be useful. For example, lysine and arginine residues can be added to the carboxy or the N-terminus of a PNA strand.

Triplex-forming molecules may further be modified to be end capped to prevent degradation using a 3′ propylamine group. Procedures for 3′ or 5′ capping oligonucleotides are well known in the art.

B. Tail Clamp

Although polypurine:polypyrimidine stretches do exist in mammalian genomes, it is desirable to target triplex formation in the absence of this requirement. The triplex-forming molecules include a “tail” added to the end of the Watson-Crick binding portion. Adding additional nucleobases, known as a “tail” or “tail clamp”, to the Watson-Crick binding portion that bind to target strand outside the triple helix further reduces the requirement for a polypurine:polypyrimidine stretch and increases the number of potential target sites. As shown in the Examples, when the triple-forming molecule is a bis-PNA, the tail is most typically added to the end of the Watson-Crick binding sequence furthest from the linker. This molecule therefore mediates a mode of binding to DNA that encompasses both triplex and duplex formation (Kaihatsu, et al., Biochemistry, 42(47):13996-4003 (2003); Bentin, et al., Biochemistry, 42(47):13987-95 (2003)). For example, if the triplex-forming molecules are tail clamp PNA (tcPNA), the PNA/DNA/PNA triple helix portion and the PNA/DNA duplex portion both produce displacement of the pyrimidine-rich strand, creating an altered helical structure that strongly provokes the nucleotide excision repair pathway and to activate the site for recombination with a donor DNA molecule (Rogers, et al., Proc. Natl. Acad. Sci. U.S.A., 99(26):16695-700 (2002)). Tail clamps added to PNAs (referred to as tcPNAs) have been described by Kaihatsu, et al., Biochemistry, 42(47):13996-4003 (2003); Bentin, et al., Biochemistry, 42(47):13987-95 (2003), and are known to bind to DNA more efficiently due to low dissociation constants. The addition of the tail also increases binding specificity and binding stringency of the triplex-forming molecules to the target duplex. It has also been found that the addition of a tail to clamp PNA improves the frequency of recombination of the donor oligonucleotide at the target site. Tail clamps can be added to any of the triplex-forming molecules described above. As demonstrated in the examples below, the addition of a 5 nucleobase tail having the sequence ATTTC to bis PNA targeting the CCR5 gene improved the induced recombination of a donor strand (2.46%) compared to the same PNA without the tail addition (0.54%).

C. Triplex-Forming Target Sequence

1. Genes to be Targeted

The triplex-forming molecules such as tcPNAs bind to a predetermined target region referred to herein as the “target sequence”, “target region”, or “target site”. The target sequence for the triplex-forming molecules is within or adjacent to a human gene that encodes a cell surface receptor for human immunodeficiency virus (HIV). The target sequence can be within the coding DNA sequence of the gene or within an intron. The target sequence can also be within DNA sequences which regulate expression of the target gene, including promoter or enhancer sequences.

Preferably, the target sequence of the triplex-forming molecules is within or is adjacent to a portion of a HIV receptor gene important to its function in HIV entry into cells, such as sequences that are involved in efficient expression of the receptor, transport of the receptor to the cell surface, stability of the receptor, viral binding by the receptor, or endocytosis of the receptor. Target sequences can be within the coding DNA sequence of the gene or within introns. Target sequences can also be within DNA sequences which regulate expression of the target gene, including promoter or enhancer sequences.

The target sequence can be within or adjacent to any gene encoding a cell surface receptor that facilitates entry of HIV into cells. The molecular mechanism of HIV entry into cells involves specific interactions between the viral envelope glycoproteins (env) and two target cell proteins, CD4 and the chemokine receptors. HIV cell tropism is determined by the specificity of the env for a particular chemokine receptor, a 7 transmembrane-spanning, G protein-coupled receptor (Steinberger, et al., Proc. Natl. Acad. Sci. USA. 97: 805-10 (2000)). The two major families of chemokine receptors are the CXC chemokine receptors and the CC chemokine receptors (CCR) so named for their binding of CXC and CC chemokines, respectively. While CXC chemokine receptors traditionally have been associated with acute inflammatory responses, the CCRs are mostly expressed on cell types found in connection with chronic inflammation and T-cell-mediated inflammatory reactions: eosinophils, basophils, monocytes, macrophages, dendritic cells, and T cells (Nansen, et al. 2002, Blood 99:4). In one embodiment, the target sequence is within or adjacent to the human genes encoding chemokine receptors, including, but not limited to, CXCR4, CCR5, CCR2b, CCR3, and CCR1.

In a preferred embodiment, the target sequence is within or adjacent to the human CCR5 gene. The CCR5 chemokine receptor is the major co-receptor for R5-tropic HIV strains, which are responsible for most cases of initial, acute HIV infection. Individuals who possess a homozygous inactivating mutation, referred to as the Δ32 mutation, in the CCR5 gene are almost completely resistant to infection by R5-tropic HIV-1 strains. The Δ32 mutation produces a 32 base pair deletion in the CCR5 coding region.

Another naturally occurring mutation in the CCR5 gene is the m303 mutation, characterized by an open reading frame single T to A base pair transversion at nucleotide 303 which indicates a cysteine to stop codon change in the first extracellular loop of the chemokine receptor protein at amino acid 101 (C101X) (Carrington et al. 1997). Mutagenesis assays have not detected the expression of the m303 co-receptor on the surface of CCR5 null transfected cells which were found to be non-susceptible to HIV-1 R5-isolates in infection assays (Blanpain, et al. (2000).

Individuals having the homozygous Δ32 inactivating mutation in the CCR5 gene display no significant adverse phenotypes, suggesting that this gene is largely dispensable for normal human health. This makes the CCR5 gene a particularly attractive target for targeted mutagenesis using the triplex-forming molecules disclosed herein. The gene for human CCR5 is known in the art and is provided at GENBANK accession number NM_{—}000579. The coding region of the human CCR5 gene is provided by nucleotides 358 to 1416 of GENBANK accession number NM_{—}000579.

2. Sequence Design

The tail-clamp bis-PNAs are designed to target a specific sequence of the target duplex nucleotide. The nucleotide sequences of the triplex-forming molecules are selected based on the sequence of the target sequence, the physical constraints, and the need to have a low dissociation constant (K_{d}) for the triplex-forming molecules/target sequence. The molecules will have a base composition which is conducive to triple-helix formation and may also take into consideration the structural motifs for third strand binding. The most stable complexes are formed on polypurine elements, however, as discussed above this requirement is reduced by the inclusion of a tail sequence on the Watson-Crick binding portion.

Preferably, the triplex-forming molecules such as tcPNAs bind to or hybridize to the target sequence under conditions of high stringency and specificity. Most preferably, the triplex-forming molecules bind in a sequence-specific manner to the target sequence. Reaction conditions for in vitro triple helix formation of triplex-forming molecules to a nucleic acid sequence vary from molecule to molecule, depending on factors such as nucleotide length, the number of G:C and A:T base pairs, and the composition of the buffer utilized in the hybridization reaction.

Typically, triplex-helix forming molecules are substantially complementary to the target sequence. Preferably, both the Watson-Crick and Hoogsteen binding portions of the triplex forming molecules are substantially complementary to the target sequence. As used herein, triplex-forming molecules are said to be substantially complementary to a target region when the molecules have a heterocyclic base composition which allows for duplex strand displacement and the formation of a triple-helix with the target region. As such, triplex-forming molecules are substantially complementary to a target region even when there are non-complementary bases present in the molecules. There are a variety of structural motifs available which can be used to determine the nucleotide sequence of the substantially complementary molecules.

Preferably, the triplex-forming molecules are between 6 and 50 nucleotides in length. The Watson-Crick portion should be 9 or more nucleobases in length, including the tail sequence. More preferably, the Watson-Crick binding portion is between about 9 and 30 nucleobases in length, including a tail sequence of between 0 and about 15 nucleobases. More preferably, the Watson-Crick binding portion is between about 10 and 25 nucleobases in length, including a tail sequence of between 0 and about 10 nucleobases. In the most preferred embodiment, the Watson-Crick binding portion is between 15 and 25 nucleobases in length, including a tail sequence of between 5 and 10 nucleobases. The Hoogsteen binding portion should be 6 or more nucleobases in length. Most preferably, the Hoogsteen binding portion is between about 6 and 15 nucleobases, inclusive.

The triplex-forming molecules are designed to target the polypurine strand of a polypurine:polypyrimidine stretch in the target duplex nucleotide. Therefore, the base composition of the triplex-forming molecules may be homopyrimidine. Alternatively, the base composition may be polypyrimidine. The addition of a “tail” reduces the requirement for polypurine:polypyrimidine run. Adding additional nucleobases, known as a “tail,” to the Watson-Crick binding portion of the triplex-forming molecules allows the Watson-Crick binding portion to bind/hybridize to the target strand outside the site of strand displacement. These additional bases reduce the requirement for the polypurine:polypyrimidine stretch in the target duplex and therefore increase the number of potential target sites. Triplex-forming oligonucleotides (TFO's) also require a polypurine:polypyrimidine to a form a triple helix. TFO's may require stretch of at least 15 and preferably 30 or more nucleotides. Peptide nucleic acids require fewer purines to a form a triple helix, although at least 10 or preferably more may be needed. Peptide nucleic acids including a tail, also referred to tail clamp PNAs, or tcPNAs, require even fewer purines to a form a triple helix. A triple helix may be formed with a target sequence containing fewer than 8 purines. Therefore, triplex-forming molecules including PNAs should be designed to target a site on duplex nucleic acid containing between 6-30 polypurine:polypyrimidines, preferably, 6-25 polypurine:polypyrimidines, more preferably 6-20 polypurine:polypyrimidines.

The addition of a “mixed-sequence” tail to the Watson-Crick-binding strand of the triplex-forming molecules such as PNAs also increases the length of the triplex-forming molecule and, correspondingly, the length of the binding site. This increases the target specificity and size of the lesion created at the target site and disrupts the helix in the duplex nucleic acid, while maintaining a low requirement for a stretch of polypurine:polypyrimidines. Increasing the length of the target sequence improves specificity for the target, for example, a target of 16 to 17 base pairs will statistically be unique in the human genome. Relative to a smaller lesion, it is likely that a larger triplex lesion with greater disruption of the underlying DNA duplex will be detected and processed more quickly and efficiently by the endogenous DNA repair machinery that facilitates recombination of the donor oligonucleotide.

In some embodiments, the target region is a polypurine site within or adjacent to a gene encoding a chemokine receptor including CXCR4, CCR5, CCR2b, CCR3, and CCR1. In a preferred embodiment, the target region is a polypurine or homopurine site within the coding region of the human CCR5 gene. Three homopurine sites in the coding region of the CCR5 gene that are especially useful as target sites for triplex-forming molecules are from positions 509-518, 679-690 and 900-908 relative to the ATG start codon.

The homopurine site from 679-690 partially encompasses the site of the nonsense mutation created by the Δ32 mutation. Triplex-forming molecules that bind to this target site are particularly useful. In one embodiment, the triplex-forming molecule is a tail clamp PNA having essentially the sequence from N-terminus to C-terminus-Lys-Lys-Lys-JTJTTJTTJT-OOO-TCTTCTTCTCATTTC-Lys-Lys-Lys (SEQ ID NO: 5), where J=pseudoisocytosine and O=flexible 8-amino-3,6-dioxaoctanoic acid, 6-aminohexanoic acid monomers, and both ends of each PNA are capped with three lysines (Lys). This dimeric tcPNA (referred to as tcPNA-679) contains two linked PNA segments and is designed to form a PNA/DNA/PNA triplex clamp on the purine-rich DNA strand of the 674 site from position 679 to 688 (inclusive) relative to the start ATG), and a DNA/PNA duplex from position 674-678 (inclusive). The ability of bis-PNAs to form such clamp structures at chromosomal targets inside cells has been previously demonstrated. The examples below using gel mobility shift assays to test the affinity of tcPNA-679 to its binding site in the CCR5 gene in vitro, which revealed strong binding by this molecule to its target site in the CCR5 gene. The examples below using allele-specific PCR also demonstrate that PNA-679 (in combination with a DNA donor containing a nonsense mutation) can induce mutation of the endogenous CCR5 gene in the human monocytic acute leukemia cell line, THP-1, k562 cells, and primary CD34+ hematopoietic stem cells. Furthermore, tcPNA-679 which contains a 5 base pair tail on the Watson-Crick binding portion induced a higher target modification frequency (2.46%) than did a clamp PNA of the same triplex-forming sequence that did not contain the additional 5 base pair tail (0.54%).

The triple-forming molecules are preferably generated using known synthesis procedures. In one embodiment, triplex-forming molecules are generated synthetically. Triplex-forming molecules can also be chemically modified using standard methods that are well known in the art.

D. Methods for Determining Triplex Formation

A useful measure of triple helix formation is the equilibrium dissociation constant, K_{d}, of the triplex, which can be estimated as the concentration of triplex-forming molecules at which triplex formation is half-maximal. Preferably, the molecules have a binding affinity for the target sequence in the range of physiologic interactions. Preferred triplex-forming molecules have a K_{d} less than or equal to approximately 10⁻⁷ M. Most preferably, the K_{d} is less than or equal to 2×10⁻⁸ M in order to achieve significant intramolecular interactions. A variety of methods are available to determine the K_{d} of triplex-forming molecules with the target duplex. In the examples which follow, the K_{d} was estimated using a gel mobility shift assay (R. H. Durland et al., Biochemistry 30, 9246 (1991)). The dissociation constant (K_{d}) can be determined as the concentration of triplex-forming molecules in which half was bound to the target sequence and half was unbound.

E. Donor Oligonucleotides

The triplex forming molecules including peptide nucleic acids may be administered in combination with, or tethered to, a donor oligonucleotide via a mixed sequence linker or used in conjunction with a non-tethered donor oligonucleotide that is substantially homologous to the target sequence. Triplex-forming molecules can induce recombination of a donor oligonucleotide sequence up to several hundred base pairs away. It is preferred that the donor oligonucleotide sequence is between 1 to 800 bases from the target binding site of the triplex-forming molecules. More preferably the donor oligonucleotide sequence is between 25 to 75 bases from the target binding site of the triplex-forming molecules. Most preferably that the donor oligonucleotide sequence is about 50 nucleotides from the target binding site of the triplex-forming molecules.

The donor sequence can contain one or more nucleic acid sequence alterations compared to the sequence of the region targeted for recombination, for example, a substitution, a deletion, or an insertion of one or more nucleotides. Successful recombination of the donor sequence results in a change of the sequence of the target region. Donor oligonucleotides are also referred to herein as donor fragments, donor nucleic acids, donor DNA, or donor DNA fragments. This strategy exploits the ability of a triplex to provoke DNA repair, potentially increasing the probability of recombination with the homologous donor DNA. It is understood in the art that a greater number of homologous positions within the donor fragment will increase the probability that the donor fragment will be recombined into the target sequence, target region, or target site. Tethering of a donor oligonucleotide to a triplex-forming molecule facilitates target site recognition via triple helix formation while at the same time positioning the tethered donor fragment for possible recombination and information transfer. Triplex-forming molecules also effectively induce homologous recombination of non-tethered donor oligonucleotides. The term “recombinagenic” as used herein, is used to define a DNA fragment, oligonucleotide, peptide nucleic acid, or composition as being able to recombine into a target site or sequence or induce recombination of another DNA fragment, oligonucleotide, or composition.

Non-tethered or unlinked fragments may range in length from 20 nucleotides to several thousand. The donor oligonucleotide molecules, whether linked or unlinked, can exist in single stranded or double stranded form. The donor fragment to be recombined can be linked or un-linked to the triplex forming molecules. The linked donor fragment may range in length from 4 nucleotides to 100 nucleotides, preferably from 4 to 80 nucleotides in length. However, the unlinked donor fragments have a much broader range, from 20 nucleotides to several thousand. In one embodiment the olignucleotide donor is between 25 and 80 nucleobases. In a further embodiment, the non-tethered donor nucleotide is about 50 to 60 nucleotides in length.

The donor oligonucleotides contain at least one mutated, inserted or deleted nucleotide relative to the target DNA sequence. The target sequence is preferably within or is adjacent to a portion of a HIV receptor gene important to its function in HIV entry into cells, such as sequences that are involved in efficient expression of the receptor, transport of the receptor to the cell surface, stability of the receptor, viral binding by the receptor, or endocytosis of the receptor. Target sequences can be within the coding DNA sequence of the gene or within introns. Target sequences can also be within DNA sequences which regulate expression of the target gene, including promoter or enhancer sequences.

The donor oligonucleotides can contain a variety of mutations relative to the target sequence. Representative types of mutations include, but are not limited to, point mutations, deletions and insertions. Deletions and insertions can result in frameshift mutations or deletions. Point mutations can cause missense or nonsense mutations. These mutations may disrupt, reduce, stop, increase, improve, or otherwise alter the expression of the target gene. Such mutations can cause one or more deficiencies in the ability of the cell surface HIV receptor to bind to HIV and allow its transport into the cell. The ultimate effect of the mutation in or adjacent to the target sequence is to inhibit or reduce the ability of the cell surface HIV receptor to bind to viral particles and permit entry of the viral particles into the cell.

Compositions including triplex-forming molecules such as tcPNA may include one or more than one donor oligonucleotides. More than one donor oligonucleotides may be administered with triplex-forming molecules in a single transfection, or sequential transfections. Use of more than one donor oligonucleotide may be useful, for example, to create a heterozygous target gene where the two alleles contain different modifications. As described in the examples below, two different antisense oriented donor oligonucleotides, donor 591 having the sequence: 5′ AT TCC CGA GTA GCA GAT GAC CAT GAC AGC TTA GGG CAG GAC CAG CCC CAA GAT GAC TAT C 3′ (SEQ ID NO: 13); and donor 597 having the sequence 5′ TT TAG GAT TCC CGA GTA GCA GAT GAC CCC TCA GAG CAG CGG CAG GAC CAG CCC CAA GAT G 3′ (SEQ ID NO: 14) can be used to induce two different non-sense mutations, one in each allele of the CCR5 gene, in the vicinity of the Δ32 deletion (mutation sites are bolded).

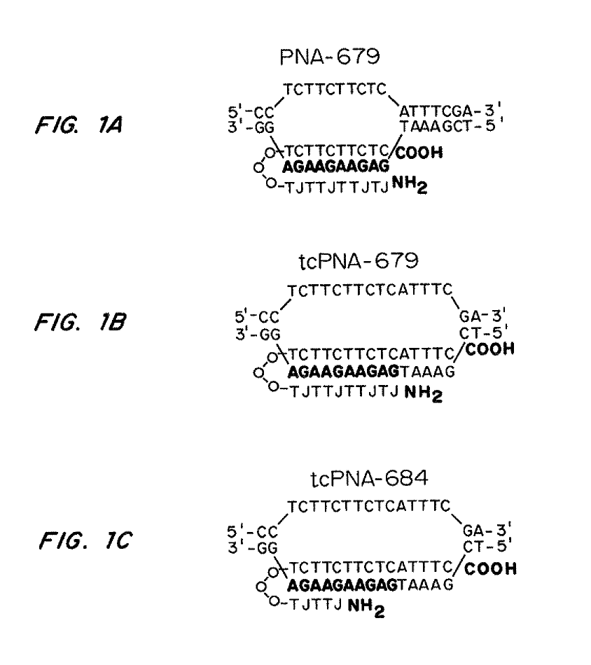

In another preferred embodiment, donor oligonucleotides are designed to span the Δ32 deletion site (as shown in FIG. 1) and induce changes into a wildtype CCR5 allele that mimic the Δ32 deletion. Donor sequences designed to target the Δ32 deletion site may be particularly usefully to facilitate knockout of the single wildtype CCR5 allele in heterozygous cells. As described in the examples below, preferred donor sequences designed to target the Δ32 deletion site include, but are not limited to,

Donor DELTA32JDC:
(SEQ ID NO: 15)
5′GATGACTATCTTTAATGTCTGGAAATTCTTCCAGAATTAATTAAGAC
TGTATGGAAAATGAGAGC 3′;
Donor DELTAJDC2:
(SEQ ID NO: 16)
5′CCCCAAGATGACTATCTTTAATGTCTGGAACGATCATCAGAATTGAT
ACTGACTGTATGGAAAATG 3′;
and
Donor DELTA32RSB:
(SEQ ID NO: 17)
5′GATGACTATCTTTAATGTCTGGAAATTCTACTAGAATTGATACTGAC
TGTATGGAAAATGAGAGC 3′.

Donor oligonucleotides are preferably DNA oligonucleotides, composed of the principal naturally-occurring nucleotides (uracil, thymine, cytosine, adenine and guanine) as the heterocyclic bases, deoxyribose as the sugar moiety, and phosphate ester linkages. Donor oligonucleotides may include modifications to nucleobases, sugar moieties, or backbone/linkages, as described above, depending on the desired structure of the replacement sequence at the site of recombination or to provide some resistance to degradation by nucleases. Modifications to the donor oligonucleotide should not prevent the donor oligonucleotide from successfully recombining at the recombination target sequence in the presence of triplex-forming molecules.

F. Methods for Determining Introduction of Alternative Sequence at the Target Site

As described in the examples below, allele-specific PCR is a preferred method for determining if a recombination event has occurred. PCR primers are designed to distinguish between the original allele, and the new predicted sequence following recombination. Other methods of determining if a recombination event has occurred are known in the art and may be selected based on the type of modification made. Methods include, but are not limited to, analysis of genomic DNA, for example by sequencing, allele-specific PCR, or restriction endonuclease selective PCR (REMS-PCR); analysis of mRNA transcribed from the target gene for example by Northern blot, in situ hybridization, real-time or quantitative reverse transcriptase (RT) PCT; and analysis of the polypeptide encoded by the target gene, for example, by immunostaining, ELISA, or FACS. In some cases, modified cells will be compared to parental controls. Other methods may include testing for changes in the function of the RNA transcribed by, or the polypeptide encoded by the target gene. For example, if the target gene encodes an enzyme, an assay designed to test enzyme function may be used.

G. Cell Targeting Moieties and Protein Transduction Domains

Formulations of the triplex-forming molecules embrace fusions of the triplex-forming molecules or modifications of the triplex-forming molecules, wherein the triplex-forming molecules are fused to another moiety or moieties. Such analogs may exhibit improved properties such as increased cell membrane permeability, activity and/or stability. Examples of moieties which may be linked or unlinked to the triplex-forming molecules or donor oligonucleotides include, for example, targeting moieties which provide for the delivery of molecules to specific cells, e.g., antibodies to hematopoietic stem cells, CD34⁺ cells, T cells or any other preferred cell type, as well as receptor and ligands expressed on the preferred cell type. Preferably, the moieties target hematopoietic stem cells. Other moieties that may be provided with the triplex-forming molecules or oligonucleotides include protein transduction domains (PTDs), which are short basic peptide sequences present in many cellular and viral proteins that mediate translocation across cellular membranes. Exemplary protein transduction domains that are well-known in the art include the Antennapedia PTD and the TAT (transactivator of transcription) PTD, poly-arginine, poly-lysine or mixtures of arginine and lysine.

H. Additional Mutagenic Agents

The triplex-forming molecules can be used alone or in combination with other mutagenic agents. As used herein, two agents are said to be used in combination when the two agents are co-administered, or when the two agents are administered in a fashion so that both agents are present within the cell or blood simultaneously. In a preferred embodiment, the additional mutagenic agents are conjugated or linked to the triplex-forming molecule. Additional mutagenic agents that can be used in combination with triplex-forming molecules include agents that are capable of directing mutagenesis, nucleic acid crosslinkers, radioactive agents, or alkylating groups, or molecules that can recruit DNA-damaging cellular enzymes. Other suitable mutagenic agents include, but are not limited to, chemical mutagenic agents such as alkylating, bialkylating or intercalating agents. A preferred agent for co-administration is psoralen-linked molecules as described in PCT/US/94/07234 by Yale University.

I. Additional Prophylactic or Therapeutic Agents

The triplex-forming molecules can be used alone or in combination with other prophylactic or therapeutic agents. As used herein, two agents are said to be used in combination when the two agents are co-administered, or when the two agents are administered in a fashion so that both agents are present within the cell or serum simultaneously. Suitable additional prophylactic or therapeutic agents include those useful to treat or prevent HIV infection. Suitable therapeutic agents include those typically used for “HAART”, which is an acronym for highly active antiretroviral therapy for the treatment of HIV-1 infection. HAART therapy typically encompasses a double nucleoside (NRTI) backbone plus either a non-nucleoside reverse transcriptase inhibitor (NNRTI) or a ritonavir pharmacologically enhanced protease inhibitor (PI/r). However the actual therapeutic composition in terms of both class and active agent varies depending upon availability of each agent and a patient's individual tolerance for each ingredient, among others. Accordingly, use of the term “HAART” is meant to broadly encompass all combinations of active therapeutic agents that the art would ascribe to this term. Exemplary HAART therapeutic agents include nucleoside & nucleotide reverse transcriptase inhibitors (NRTI), non-nucleoside reverse transcriptase inhibitors (nNRTI), protease inhibitors, integrase inhibitors, entry inhibitors and maturation inhibitors.

Suitable entry inhibitors include other therapeutic agents that function as antagonists to HIV cell surface receptors, including CCR5. CCR5 antagonists include small molecule noncompetitive allosteric antagonists which bind in a cavity formed between several transmembrane helices of the CCR5 protein, including, but not limited to, TAK-779, TAK-220, TAK-652, aplaviroc, maraviroc and vicroviroc.

It may also be desirable to administer compositions containing triplex-forming molecules in combination with agents that further enhance the frequency of gene modification in cells. For example, the disclosed compositions can be administered in combination with a histone deacetylase (HDAC) inhibitor, such as suberoylanilide hydroxamic acid (SAHA), which has been found to promote increased levels of gene targeting in asynchronous cells. The nucleotide excision repair pathway is also known to facilitate triplex-forming molecule-mediated recombination. Therefore, the disclosed compositions can be administered in combination with an agent that enhances or increases the nucleotide excision repair pathway, for example an agent that increases the expression, or activity, or localization to the target site, of the endogenous damage recognition factor XPA. Compositions may also be administered in combination with a second active agent that enhances uptake or delivery of the triplex-forming molecules or the donor oligonucleotides. For example, the lysosomotropic agent chloroquine has been shown to enhance delivery of PNAs into cells (Abes, et al., J. Controll. Rel., 110:595-604 (2006). In some embodiments, the triplex-forming molecules and/or the donor oligonucleotides can be package in PLGA micro or nanoparticles. Agents that improve the frequency of gene modification are particularly useful for in vitro and ex vivo application, for example ex vivo modification of hematopoietic stem cells for therapeutic use.

### II. Methods of Use

A. Inactivation of Cell Surface Receptors for HIV

Triplex-forming molecules bind/hybridize to a target sequence within or adjacent to a human gene encoding a cell surface receptor for HIV, displacing the polyprimidine strand, forming a triplex structure and hybrid duplex with the polypurine strand. The binding of triplex-forming molecules to the target region stimulates mutations within or adjacent target region using cellular DNA synthesis, recombination, and repair mechanisms. In targeted recombination, triplex forming molecules are administered to a cell in combination with a separate donor oligonucleotide fragment which minimally contains a sequence substantially complementary to the target region or a region adjacent to the target region, referred to herein as the donor fragment. The donor fragment can further contain nucleic acid sequences which are to be inserted within the target region. The co-administration of triplex-forming molecules with the fragment to be recombined increases the frequency of insertion of the donor fragment within the target region when compared to procedures which do not employ triplex forming molecules.

The triplex-forming molecules in combination with the donor oligonucleotides induce site-specific mutations or alterations of the nucleic acid sequence within or adjacent to the target sequence. The target sequence is preferably within or is adjacent to a portion of a HIV receptor gene important to its function in HIV entry into cells, such as sequences that are involved in efficient expression of the receptor, transport of the receptor to the cell surface, stability of the receptor, viral binding by the receptor, or endocytosis of the receptor. Target sequences can be within the coding DNA sequence of the gene or within introns. Target sequences can also be within DNA sequences which regulate expression of the target gene, including promoter or enhancer sequences.

The triplex-forming molecules in conjunction with donor oligonucleotides can induce any of a range of mutations in or adjacent to the target sequence. Representative types of mutations include, but are not limited to, point mutations, deletions and insertions. Point mutations can cause missense or nonsense mutations. Deletions and insertions can result in frameshift mutations or deletions. Such mutations can cause one or more deficiencies in the ability of the cell surface HIV receptor to bind to HIV and allow its transport into the cell. For example, mutations can result in reduced expression (transcription and/or translation) of the target gene. Mutations can also result in a defect in the transport of the receptor to the cell surface or a reduction in the stability of the protein such that its presentation at the cell surface is reduced or inhibited. Mutations can also reduce the ability of the receptor to be internalized by endocytosis, or to be routed through proper endocytic pathways. Mutations can also reduce or inhibitor binding of HIV viral particles by the cell surface receptor.

The ultimate effect of the mutation in or adjacent to the target sequence is to inhibit or reduce the ability of the cell surface HIV receptor to bind to viral particles and permit entry of the viral particles into the cell. The particular HIV cell surface receptor gene targeted by the triplex-forming molecule determines which strains of HIV will display reduced or inhibited binding and entry into the cell. HIV-1 isolates exhibit marked differences in their ability to infect CD4⁺ T cells. While all strains infect primary CD4⁺ T cells, most primary isolates also infect macrophages (M tropic) but fail to infect transformed CD4⁺ T cell lines. Other isolates replicate well in CD4⁺ T cell lines (T tropic) but fail to infect macrophages. The underlying source of permissiveness for M and T tropic viruses is determined by the co-receptor used by the HIV strains. CCR5 confers susceptibility to infection by certain M-tropic (R5-tropic) strains of HIV-1, whereas CXCR4, serves as a cofactor for T tropic (X4-tropic) HIV-1 strains. Thus, mutations in the CCR5 gene can create cells that are R5-tropic virus-resistant cells, and mutations in the CXCR4 gene can create cells that are X4-tropic virus-resistant cells. In some embodiments, more than one species of triplex-forming molecules is used to induce mutations in more than one cell surface HIV receptor. This can result in cells that are resistant to HIV strains with more than one tropism.

In one embodiment, the compositions and methods disclosed herein are used to cause mutations in the human CCR5 gene. In a preferred embodiment, the mutation mimics a naturally occurring polymorphism in the human CCR5 gene that causes a 32 basepair deletion of the CCR5 receptor referred to commonly in the art as the CCR5 Δ32 mutation. This mutation causes a frameshift and deletion of the last three transmembrane domains of the CCR5 protein. In some embodiments the compositions and methods disclosed herein are used to target the Δ32 mutation site to facilitate knockout of the wildtype CCR5 allele.

B. Generation of HIV Receptor Mutant Cell Lines

Compositions containing triplex-forming molecules and methods disclosed herein are useful for the generation of cell lines containing a diverse range of mutations in genes encoding cell surface HIV receptors. Cell lines can contain mutations in or adjacent to one or more genes encoding cell surface receptors and/or can contain one or more mutations in or adjacent to a single gene encoding a cell surface receptor for HIV. For example, the compositions may be used to identify new gene alterations confer resistance to infections, including viral infections such as HIV. First targeted mutations are introduced into the cells in vitro (or ex vivo) using the disclosed compositions, as described below and the in the examples. Next the modified cells can be tested for resistance to infection. As described in the examples below, modified cells can be challenged with HIV, such as R5-tropic HIV-1 in parallel with unmodified, parental cells. At various time points following infection, supernatants from the cells can be harvested and analyzed for core protein p24 antigen levels. Reduced p24 levels in modified cells compared to parental cells indicates a decrease in HIV infection.

Such cell lines are also useful for the screening and development of other HIV therapeutic agents, including other agents that inhibit or reduce the entry of HIV into a cell. Any cell that expresses at least one cell surface receptor for HIV and that is capable of being transfected or transduced with compositions containing triplex-forming molecules can be used, including primary isolated cells and immortalized cell lines. The cells are preferably hematopoietic in origin and can be hematopoietic stem cells. Other suitable hematopoietic cells include T cells. T cells include all cells which express CD3, including T cell subsets which also express CD4 and CD8. T cells include both naive and memory cells and effector cells such as CTL. T-cells also include regulatory cells such as Th1, Tc1, Th2, Tc2, Th3, Treg, and Tr1 cells. T cells used for generation of cell lines containing mutations in genes encoding cell surface HIV receptors are preferably CD4⁺ T cells.

C. Methods of Use as a Molecular Research Tool

For in vitro research studies, a solution containing the triplex-forming molecules is added directly to a solution containing the DNA molecules of interest in accordance with methods well known to those skilled in the art and described in more detail in the examples below.

In vivo research studies are conducted by transfecting cells with the triplex-forming molecules and optionally one or more donor oligonucleotides in a solution such as growth media with the transfected cells for a sufficient amount of time for entry of the triplex-forming molecules into the cells for triplex formation with a target duplex sequence. Cells may transfected by electroporation, as described in the example below, or through any other suitable means known in the art. The target duplex sequence may be episomal DNA, such as nonintegrated plasmid DNA. The target duplex sequence may also be exogenous DNA, such as plasmid DNA or DNA from a viral construct, which has been integrated into the cell's chromosomes. The target duplex sequence may also be a sequence endogenous to the cell. The transfected cells may be in suspension or in a monolayer attached to a solid phase, or may be cells within a tissue wherein the triplex-forming molecules are in the extracellular fluid.

D. Treatment of Subjects with or at Risk of Developing an HIV Infection

In general, the compositions and methods described herein are useful for treating a subject having or being predisposed to HIV infection. The compositions are useful as prophylactic compositions, which confer resistance in a subject to HIV. The compositions are also useful as therapeutic compositions, which can be used to initiate or enhance a subject's resistance to HIV infection. The compositions and methods generate CD4⁺ immune cells which are resistant to infection by HIV by altering the expression, localization, stability, binding activity and/or endocytosis of at least one cell surface receptor for HIV. The result of treatment with the compositions and methods disclosed herein is to prevent infection of an individual with HIV or to reduce the viral load in a subject that is already infected with HIV. Another result of treatment can be an increase in CD4 counts in subjects infected with HIV. Methods for assessing HIV viral load and CD4 counts are well known in the art.

Preferably, the compositions and methods described herein can be used to treat or prevent any disease or condition that arises from HIV infection, such as AIDS and ARC. It should be recognized that the methods disclosed herein can be practiced in conjunction with existing antiviral therapies to effectively treat or prevent HIV infection and diseases and conditions that arise from HIV infection.

1. Ex Vivo Gene Therapy for Treating or Preventing Genetic Disorders

In one embodiment, ex vivo gene therapy of cells is used for the treatment or prevention of HIV infection in a subject. For ex vivo prophylaxis or therapy of HIV infection, cells are isolated from a subject and contacted ex vivo with the compositions disclosed herein to produce cells containing mutations in or adjacent to genes encoding HIV cell surface receptors including, but not limited to, CXCR4, CCR5, CCR2b, CCR3, and CCR1. In a preferred embodiment, the cells are isolated from the subject to be treated or from a syngenic host. Target cells are removed from a subject prior to contacting with triplex-forming molecules and donor oligonucleotides. The cells can be hematopoietic progenitor or stem cells. In a preferred embodiment, the target cells are CD34⁺ hematopoietic stem cells. CD34⁺ hematopoietic stem cells have been shown to be resistant to HIV infection. The resistance of CD34⁺ cells to HIV infection makes them an especially attractive cell type for gene therapy of HIV using the compositions and methods disclosed herein because they can be taken from HIV infected individuals and mutated without fear of HIV contamination. CD34+ cells can be isolated from a patient, the target gene (such as CCR5) altered or repaired ex-vivo using the disclosed compositions and methods, and the cells reintroduced back into the patient as a treatment or a cure.

Such stem cells can be isolated and enriched by one of skill in the art. Methods for such isolation and enrichment of CD34⁺ and other cells are known in the art and disclosed for example in U.S. Pat. Nos. 4,965,204; 4,714,680; 5,061,620; 5,643,741; 5,677,136; 5,716,827; 5,750,397 and 5,759,793. As used herein in the context of compositions enriched in hematopoietic progenitor and stem cells, “enriched” indicates a proportion of a desirable element (e.g. hematopoietic progenitor and stem cells) which is higher than that found in the natural source of the cells. A composition of cells may be enriched over a natural source of the cells by at least one order of magnitude, preferably two or three orders, and more preferably 10, 100, 200 or 1000 orders of magnitude.

In humans, CD34⁺ cells can be recovered from cord blood, bone marrow or from blood after cytokine mobilization effected by injecting the donor with hematopoietic growth factors such as granulocyte colony stimulating factor (G-CSF), granulocyte-monocyte colony stimulating factor (GM-CSF), stem cell factor (SCF) subcutaneously or intravenously in amounts sufficient to cause movement of hematopoietic stem cells from the bone marrow space into the peripheral circulation. Initially, bone marrow cells may be obtained from any suitable source of bone marrow, e.g. tibiae, femora, spine, and other bone cavities. For isolation of bone marrow, an appropriate solution may be used to flush the bone, which solution will be a balanced salt solution, conveniently supplemented with fetal calf serum or other naturally occurring factors, in conjunction with an acceptable buffer at low concentration, generally from about 5 to 25 mM. Convenient buffers include Hepes, phosphate buffers, lactate buffers, etc.

Cells can be selected by positive and negative selection techniques. Cells can be selected using commercially available antibodies which bind to hematopoietic progenitor or stem cell surface antigens, e.g. CD34, using methods known to those of skill in the art. For example, the antibodies may be conjugated to magnetic beads and immunogenic procedures utilized to recover the desired cell type. Other techniques involve the use of fluorescence activated cell sorting (FACS). The CD34 antigen, which is found on progenitor cells within the hematopoietic system of non-leukemic individuals, is expressed on a population of cells recognized by the monoclonal antibody My-10 (i.e., express the CD34 antigen) and can be used to isolate stem cell for bone marrow transplantation. My-10, deposited with the American Type Culture Collection (Rockville, Md.) as HB-8483, is commercially available as anti-HPCA 1. Additionally, negative selection of differentiated and “dedicated” cells from human bone marrow can be utilized, to select against substantially any desired cell marker. For example, progenitor or stem cells, most preferably CD34⁺ cells, can be characterized as being any of CD3⁻, CD7⁻, CD8⁻, CD10⁻, CD14⁻, CD15⁻, CD19⁻, CD20⁻, CD33⁻, Class II HLA⁺ and Thy-1⁺.

Once progenitor or stem cells have been isolated, they may be propagated by growing in any suitable medium. For example, progenitor or stem cells can be grown in conditioned medium from stromal cells, such as those that can be obtained from bone marrow or liver associated with the secretion of factors, or in medium comprising cell surface factors supporting the proliferation of stem cells. Stromal cells may be freed of hematopoietic cells employing appropriate monoclonal antibodies for removal of the undesired cells.

The isolated cells are contacted ex vivo with a combination of triplex-forming molecules and donor oligonucleotides in amounts effective to cause the desired mutations in or adjacent to genes encoding cell surface receptors for HIV. These cells are referred to herein as modified cells. Methods for transfection of cells with oligonucleotides and peptide nucleic acids are well known in the art (Koppelhus, et al., Adv. Drug Deliv. Rev., 55(2): 267-280 (2003)). It may be desirable to synchronize the cells in S-phase to further increase the frequency of gene correction. Methods for synchronizing cultured cells, for example by double thymidine block, are known in the art (Zielke, et al., Methods Cell Biol., 8:107-121 (1974)).

The modified cells can be maintained or expanded in culture prior to administration to a subject. Culture conditions are generally known in the art depending on the cell type. Conditions for the maintenance of CD34⁺ in particular have been well studied, and several suitable methods are available. A common approach to ex vivo multi-potential hematopoietic cell expansion is to culture purified progenitor or stem cells in the presence of early-acting cytokines such as interleukin-3. It has also been shown that inclusion, in a nutritive medium for maintaining hematopoietic progenitor cells ex vivo, of a combination of thrombopoietin (TPO), stem cell factor (SCF), and flt3 ligand (Flt-3L; i.e., the ligand of the flt3 gene product) was useful for expanding primitive (i.e., relatively non-differentiated) human hematopoietic progenitor cells in vitro, and that those cells were capable of engraftment in SCID-hu mice (Luens et al., 1998, Blood 91:1206-1215). In other known methods, cells can be maintained ex vivo in a nutritive medium (e.g., for minutes, hours, or 3, 6, 9, 13, or more days) comprising murine prolactin-like protein E (mPLP-E) or murine prolactin-like protein F (mPIP-F; collectively mPLP-E/IF) (U.S. Pat. No. 6,261,841). It will be appreciated that other suitable cell culture and expansion method can be used in accordance with the invention as well. Cells can also be grown in serum-free medium, as described in U.S. Pat. No. 5,945,337.

In another embodiment, the modified hematopoietic stem cells are differentiated ex vivo into CD4⁺ cells culture using specific combinations of interleukins and growth factors prior to administration to a subject using methods well known in the art. The cells may be expanded ex vivo in large numbers, preferably at least a 5-fold, more preferably at least a 10-fold and even more preferably at least a 20-fold expansion of cells compared to the original population of isolated hematopoietic stem cells.

In another embodiment cells for ex vivo gene therapy, the cells to be used can be dedifferentiated somatic cells. Somatic cells can be reprogrammed to become pluripotent stem-like cells that can be induced to become hematopoietic progenitor cells. The hematopoietic progenitor cells can then be treated with triplex-forming molecules and donor oligonucleotides as described above with respect to CD34⁺ cells to produce recombinant immune cells that do not express functional receptors involved in HIV infection. Representative somatic cells that can be reprogrammed include, but are not limited to fibroblasts, adipocytes, and muscles cells. Hematopoietic progenitor cells from induced stem-like cells have been successfully developed in the mouse (Hanna, J. et al. Science, 318:1920-1923 (2007)).

To produce hematopoietic progenitor cells from induced stem-like cells, somatic cells are harvested from a host. In a preferred embodiment, the somatic cells are autologous fibroblasts. The cells are cultured and transduced with vectors encoding Oct4, Sox2, Klf4, and c-Myc transcription factors. The transduced cells are cultured and screened for embryonic stem cell (ES) morphology and ES cell markers including, but not limited to AP, SSEA1, and Nanog. The transduced ES cells are cultured and induced to produce induced stem-like cells. Cells are then screened for CD41 and c-kit markers (early hematopoietic progenitor markers) as well as markers for myeloid and erythroid differentiation.

The modified hematopoietic stem cells or modified induced hematopoietic progenitor cells are then introduced into a subject. Delivery of the cells may be effected using various methods and includes most preferably intravenous administration by infusion as well as direct depot injection into periosteal, bone marrow and/or subcutaneous sites.

The subject receiving the modified cells may be treated for bone marrow conditioning to enhance engraftment of the cells. The recipient may be treated to enhance engraftment, using a radiation or chemotherapeutic treatment prior to the administration of the cells. Upon administration, the cells will generally require a period of time to engraft. Achieving significant engraftment of hematopoietic stem or progenitor cells typically takes a period week to months.

A high percentage of engraftment of modified hematopoietic stem cells is not envisioned to be necessary to achieve significant prophylactic or therapeutic effect. It is expected that the engrafted cells will expand over time following engraftment to increase the percentage of modified cells. The modified cells are resistant to infection by HIV relative to unmodified cells due to altered expression, localization, stability, binding activity and/or endocytosis of at least one cell surface receptor for HIV. Therefore, in a subject with an HIV infection, the modified cells are expected to have a competitive advantage over non-modified cells. It is expected that engraftment of only a small number or small percentage of modified hematopoietic stem cells will be required to provide a prophylactic or therapeutic effect.

In preferred embodiments, the cells to be administered to a subject will be autologous, e.g. derived from the subject, or syngenic. Nevertheless, allogeneic cell transplants are also envisioned, and allogeneic bone marrow transplants are carried out routinely. Allogeneic cell transplantation can be offered to those patients who lack an appropriate sibling donor by using bone marrow from antigenically matched, genetically unrelated donors (identified through a national registry), or by using hematopoietic progenitor or stem-cells obtained or derived from a genetically related sibling or parent whose transplantation antigens differ by one to three of six human leukocyte antigens from those of the patient.

2. In Vivo Gene Therapy

In another embodiment, the triplex-forming molecules are administered directly to a subject with or having been predisposed to HIV infection.

a. Formulations

The disclosed compositions including triplex-forming molecules such as tcPNAs and one or more donor fragments are preferably employed for therapeutic uses in combination with a suitable pharmaceutical carrier. Such compositions include an effective amount of triplex-forming molecules and donor fragment, and a pharmaceutically acceptable carrier or excipient. An effective amount of triplex-forming molecules may be enough molecules to induce strand displacement and formation of a triple helix at the target site. An effective amount of triplex-forming molecules may also be an amount effective to increase the rate of recombination of a donor fragment relative to administration of the donor fragment in the absence of triplex-forming molecules. Compositions should include an amount of donor fragment effective to recombine at the target site in the presence of triplex-forming molecules. The formulation is made to suit the mode of administration. Pharmaceutically acceptable carriers are determined in part by the particular composition being administered, as well as by the particular method used to administer the composition. Accordingly, there is a wide variety of suitable formulations of pharmaceutical compositions containing the nucleic acids.

It is understood by one of ordinary skill in the art that nucleotides administered in vivo are taken up and distributed to cells and tissues (Huang, et al., FEBS Lett., 558(1-3):69-73 (2004)). For example, Nyce, et al. have shown that antisense oligodeoxynucleotides (ODNs) when inhaled bind to endogenous surfactant (a lipid produced by lung cells) and are taken up by lung cells without a need for additional carrier lipids (Nyce, et al., Nature, 385:721-725 (1997)). Small nucleic acids are readily taken up into T24 bladder carcinoma tissue culture cells (Ma, et al., Antisense Nucleic Acid Drug Dev., 8:415-426 (1998)).

The disclosed compositions including triplex-forming molecules, such as tcPNAs and donor fragments may be in a formulation for administration topically, locally or systemically in a suitable pharmaceutical carrier. Remington's Pharmaceutical Sciences, 15th Edition by E. W. Martin (Mark Publishing Company, 1975), discloses typical carriers and methods of preparation. The compound may also be encapsulated in suitable biocompatible microcapsules, microparticles, nanoparticles, or microspheres formed of biodegradable or non-biodegradable polymers or proteins or liposomes for targeting to cells. Such systems are well known to those skilled in the art and may be optimized for use with the appropriate nucleic acid.

Various methods for nucleic acid delivery are described, for example, in Sambrook et al., Molecular Cloning: A Laboratory Manual, Cold Spring Harbor Laboratory, New York (1989); and Ausubel et al., Current Protocols in Molecular Biology, John Wiley & Sons, New York (1994). Such nucleic acid delivery systems comprise the desired nucleic acid, by way of example and not by limitation, in either “naked” form as a “naked” nucleic acid, or formulated in a vehicle suitable for delivery, such as in a complex with a cationic molecule or a liposome forming lipid, or as a component of a vector, or a component of a pharmaceutical composition. The nucleic acid delivery system can be provided to the cell either directly, such as by contacting it with the cell, or indirectly, such as through the action of any biological process. The nucleic acid delivery system can be provided to the cell by endocytosis, receptor targeting, coupling with native or synthetic cell membrane fragments, physical means such as electroporation, combining the nucleic acid delivery system with a polymeric carrier such as a controlled release film or nanoparticle or microparticle, using a vector, injecting the nucleic acid delivery system into a tissue or fluid surrounding the cell, simple diffusion of the nucleic acid delivery system across the cell membrane, or by any active or passive transport mechanism across the cell membrane. Additionally, the nucleic acid delivery system can be provided to the cell using techniques such as antibody-related targeting and antibody-mediated immobilization of a viral vector.

Formulations for topical administration may include ointments, lotions, creams, gels, drops, suppositories, sprays, liquids and powders. Conventional pharmaceutical carriers, aqueous, powder or oily bases, or thickeners can be used as desired.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions, solutions or emulsions that can include suspending agents, solubilizers, thickening agents, dispersing agents, stabilizers, and preservatives. Formulations for injection may be presented in unit dosage form, e.g., in ampules or in multi-dose containers, optionally with an added preservative. The compositions may take such forms as sterile aqueous or nonaqueous solutions, suspensions and emulsions, which can be isotonic with the blood of the subject in certain embodiments. Examples of nonaqueous solvents are polypropylene glycol, polyethylene glycol, vegetable oil such as olive oil, sesame oil, coconut oil, arachis oil, peanut oil, mineral oil, injectable organic esters such as ethyl oleate, or fixed oils including synthetic mono or di-glycerides. Aqueous carriers include water, alcoholic/aqueous solutions, emulsions or suspensions, including saline and buffered media. Parenteral vehicles include sodium chloride solution, 1,3-butandiol, Ringer's dextrose, dextrose and sodium chloride, lactated Ringer's or fixed oils. Intravenous vehicles include fluid and nutrient replenishers, and electrolyte replenishers (such as those based on Ringer's dextrose). Preservatives and other additives may also be present such as, for example, antimicrobials, antioxidants, chelating agents and inert gases. In addition, sterile, fixed oils are conventionally employed as a solvent or suspending medium. For this purpose any bland fixed oil including synthetic mono- or di-glycerides may be employed. In addition, fatty acids such as oleic acid may be used in the preparation of injectables. Carrier formulation can be found in Remington's Pharmaceutical Sciences, Mack Publishing Co., Easton, Pa. Those of skill in the art can readily determine the various parameters for preparing and formulating the compositions without resort to undue experimentation.

The triplex-forming molecules alone or in combination with other suitable components, can also be made into aerosol formulations (i.e., they can be “nebulized”) to be administered via inhalation. Aerosol formulations can be placed into pressurized acceptable propellants, such as dichlorodifluoromethane, propane, nitrogen, and air. For administration by inhalation, the compounds are delivered in the form of an aerosol spray presentation from pressurized packs or a nebulizer, with the use of a suitable propellant.

In some embodiments, the triplex-forming molecules and donor oligonucleotides described above may include pharmaceutically acceptable carriers with formulation ingredients such as salts, carriers, buffering agents, emulsifiers, diluents, excipients, chelating agents, fillers, drying agents, antioxidants, antimicrobials, preservatives, binding agents, bulking agents, silicas, solubilizers, or stabilizers. In one embodiment, the triplex-forming molecules and/or donors are conjugated to lipophilic groups like cholesterol and lauric and lithocholic acid derivatives with C32 functionality to improve cellular uptake. For example, cholesterol has been demonstrated to enhance uptake and serum stability of siRNA in vitro (Lorenz, et al., Bioorg. Med. Chem. Lett., 14(19):4975-4977 (2004)) and in vivo (Soutschek, et al., Nature, 432(7014):173-178 (2004)). In addition, it has been shown that binding of steroid conjugated oligonucleotides to different lipoproteins in the bloodstream, such as LDL, protect integrity and facilitate biodistribution (Rump, et al., Biochem. Pharmacol., 59(11):1407-1416 (2000)). Other groups that can be attached or conjugated to the compound described above to increase cellular uptake, include acridine derivatives; cross-linkers such as psoralen derivatives, azidophenacyl, proflavin, and azidoproflavin; artificial endonucleases; metal complexes such as EDTA-Fe(II) and porphyrin-Fe(II); alkylating moieties; nucleases such as alkaline phosphatase; terminal transferases; abzymes; cholesteryl moieties; lipophilic carriers; peptide conjugates; long chain alcohols; phosphate esters; radioactive markers; non-radioactive markers; carbohydrates; and polylysine or other polyamines. U.S. Pat. No. 6,919,208 to Levy, et al., also describes methods for enhanced delivery. These pharmaceutical formulations may be manufactured in a manner that is itself known, e.g., by means of conventional mixing, dissolving, granulating, levigating, emulsifying, encapsulating, entrapping or lyophilizing processes.

b. Methods of Administration

In general, methods of administering compounds, including oligonucleotides and related molecules, are well known in the art. In particular, the routes of administration already in use for nucleic acid therapeutics, along with formulations in current use, provide preferred routes of administration and formulation for the triplex-forming molecules described above. Preferably the triplex-forming molecules and donor oligonucleotides are injected into the organism undergoing genetic manipulation, such as an animal requiring gene therapy for the treatment or prevention of HIV infection.

The disclosed compositions including triplex-forming molecules and donor oligonucleotides can be administered by a number of routes including, but not limited to: oral, intravenous, intraperitoneal, intramuscular, transdermal, subcutaneous, topical, sublingual, or rectal means. The preferred route of administration is intravenous. Triplex-forming molecules and oligonucleotides can also be administered via liposomes. Such administration routes and appropriate formulations are generally known to those of skill in the art.

Administration of the formulations may be accomplished by any acceptable method which allows the triplex-forming molecules and a donor nucleotide, to reach their targets.

Any acceptable method known to one of ordinary skill in the art may be used to administer a formulation to the subject. The administration may be localized (i.e., to a particular region, physiological system, tissue, organ, or cell type) or systemic, depending on the condition being treated.

Injections can be e.g., intravenous, intradermal, subcutaneous, intramuscular, or intraperitoneal. In some embodiments, the injections can be given at multiple locations. Implantation includes inserting implantable drug delivery systems, e.g., microspheres, hydrogels, polymeric reservoirs, cholesterol matrixes, polymeric systems, e.g., matrix erosion and/or diffusion systems and non-polymeric systems, e.g., compressed, fused, or partially-fused pellets. Inhalation includes administering the composition with an aerosol in an inhaler, either alone or attached to a carrier that can be absorbed. For systemic administration, it may be preferred that the composition is encapsulated in liposomes.

The triplex-forming molecules and donor oligonucleotide may be delivered in a manner which enables tissue-specific uptake of the agent and/or nucleotide delivery system. Techniques include using tissue or organ localizing devices, such as wound dressings or transdermal delivery systems, using invasive devices such as vascular or urinary catheters, and using interventional devices such as stents having drug delivery capability and configured as expansive devices or stent grafts.

The formulations may be delivered using a bioerodible implant by way of diffusion or by degradation of the polymeric matrix. In certain embodiments, the administration of the formulation may be designed so as to result in sequential exposures to the triplex-forming molecules, and donor oligonucleotides, over a certain time period, for example, hours, days, weeks, months or years. This may be accomplished, for example, by repeated administrations of a formulation or by a sustained or controlled release delivery system in which the compositions are delivered over a prolonged period without repeated administrations. Administration of the formulations using such a delivery system may be, for example, by oral dosage forms, bolus injections, transdermal patches or subcutaneous implants. Maintaining a substantially constant concentration of the composition may be preferred in some cases.

Other delivery systems suitable include time-release, delayed release, sustained release, or controlled release delivery systems. Such systems may avoid repeated administrations in many cases, increasing convenience to the subject and the physician. Many types of release delivery systems are available and known to those of ordinary skill in the art. They include, for example, polymer-based systems such as polylactic and/or polyglycolic acids, polyanhydrides, polycaprolactones, copolyoxalates, polyesteramides, polyorthoesters, polyhydroxybutyric acid, and/or combinations of these. Microcapsules of the foregoing polymers containing nucleic acids are described in, for example, U.S. Pat. No. 5,075,109. Other examples include non-polymer systems that are lipid-based including sterols such as cholesterol, cholesterol esters, and fatty acids or neutral fats such as mono-, di- and triglycerides; hydrogel release systems; liposome-based systems; phospholipid based-systems; silastic systems; peptide based systems; wax coatings; compressed tablets using conventional binders and excipients; or partially fused implants. Specific examples include erosional systems in which the oligonucleotides are contained in a formulation within a matrix (for example, as described in U.S. Pat. Nos. 4,452,775, 4,675,189, 5,736,152, 4,667,013, 4,748,034 and 5,239,660), or diffusional systems in which an active component controls the release rate (for example, as described in U.S. Pat. Nos. 3,832,253, 3,854,480, 5,133,974 and 5,407,686). The formulation may be as, for example, microspheres, hydrogels, polymeric reservoirs, cholesterol matrices, or polymeric systems. In some embodiments, the system may allow sustained or controlled release of the composition to occur, for example, through control of the diffusion or erosion/degradation rate of the formulation containing the triplex-forming molecules and donor oligonucleotides. In addition, a pump-based hardware delivery system may be used to deliver one or more embodiments.

Examples of systems in which release occurs in bursts include systems in which the composition is entrapped in liposomes which are encapsulated in a polymer matrix, the liposomes being sensitive to specific stimuli, e.g., temperature, pH, light or a degrading enzyme and systems in which the composition is encapsulated by an ionically-coated microcapsule with a microcapsule core degrading enzyme. Examples of systems in which release of the inhibitor is gradual and continuous include, e.g., erosional systems in which the composition is contained in a form within a matrix and effusional systems in which the composition permeates at a controlled rate, e.g., through a polymer. Such sustained release systems can be in the form of pellets, or capsules.

Use of a long-term release implant may be particularly suitable in some embodiments. “Long-term release,” as used herein, means that the implant containing the composition is constructed and arranged to deliver therapeutically effective levels of the composition for at least 30 or 45 days, and preferably at least 60 or 90 days, or even longer in some cases. Long-term release implants are well known to those of ordinary skill in the art, and include some of the release systems described above.

Compositions including triplex-forming molecules and donor oligonucleotides and methods of their use will be further understood in view of the following non-limiting example.

### EXAMPLES

### Example 1

### Sequence Specific PNAs Bind the CCR5 Gene on a Plasmid Substrate

Materials and Methods

Design and Synthesis of PNAs and Donor Oligonucleotides

PNA-679 (sequence from N-terminus to C-terminus-Lys-Lys-Lys-JTJTTJTTJT-OOO-TCTTCTTCTC-Lys-Lys-Lys (SEQ ID NO: 3)); tcPNA-679 (sequence from N-terminus to C-terminus-Lys-Lys-Lys-JTJTTJTTJT-OOO-TCTTCTTCTCATTTC-Lys-Lys-Lys (SEQ ID NO: 5)), and tcPNA-684 (sequence from N-terminus to C-terminus-Lys-Lys-Lys-JTTJT-OOO-TCTTCTTCTCATTTC-Lys-Lys-Lys (SEQ ID NO: 7)) to the polypurine target site in CCR5. J=pseudoisocytosine. Three lysine residues were conjugated to both the N and C terminal ends of the PNA for increased bioactivity and 8-amino-2,6-dioxaoctanoic acid linkers were used as the flexible linker “O.” PNAs are depicted in FIGS. 1A-1C without 3× lysine caps at each end.

DNA oligonucleotides were synthesized by the Midland Certified Reagent Company Inc. (Midland, Tex.) and purified by RP-HPLC. The sequences of the DNA oligonucleotides are (mutation sites in bold) donor 591: 5′ AT TCC CGA GTA GCA GAT GAC CAT GAC AGC TTA GGG CAG GAC CAG CCC CAA GAT GAC TAT C 3′ (SEQ ID NO: 13) and donor 597: 5′ TT TAG GAT TCC CGA GTA GCA GAT GAC CCC TCA GAG CAG CGG CAG GAC CAG CCC CAA GAT G 3′ (SEQ ID NO: 14). All oligonucleotides were 5′ and 3′ end protected with three phosphorothioate internucleoside linkages.

Electrophoretic Mobility Shift Assays

To test the binding of tcPNA-679 to the CCR5 target site, increasing concentrations of tcPNA-679 were incubated with 2 μg plasmid DNA containing the target site in KCl to a final concentration of 10 μM; TE was added to a final volume of 10 μL, and the reactions were allowed to incubate at 37° C. overnight. Samples were digested with restriction enzymes flanking the binding site, and the products were analyzed by electrophoresis on an 8% non-denaturing PAGE gel. Silver stain was used for visualization.

Results

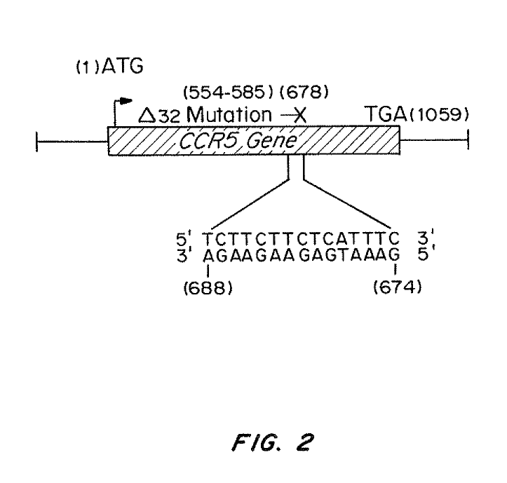

A series of triplex-forming PNAs were designed to bind to the CCR5 gene at a polypurine stretch encompassing positions 679-688 (FIG. 2). PNA-679 forms a PNA clamp with Watson-Crick and Hoogsteen binding domains of equal length (FIGS. 1A-C), similar to PNA clamp designs reported previously (Chin et al., Proc. Natl. Acad. Sci. USA, 105:13514-9 (2008)). To extend the length of the recognition site beyond the homopurine sequence, a series of “tail-clamp” PNAs (tcPNAs) were tested (Bentin, et al., Biochemistry 42(47):13987-95 (2003)). tcPNA-679 binds to form a PNA/DNA/PNA triple helix “clamp” within the polypurine stretch at positions 679-688 of the CCR5 gene and includes an additional 5-bp “tail” forming a PNA/DNA duplex at positions 674-678 (FIG. 1). This molecule mediates a mode of PNA binding to DNA that encompasses both triplex and duplex formation and in doing so targets a unique 15-bp sequence in the CCR5 gene (Bentin, et al., Biochemistry 42(47):13987-95 (2003), Kaihatsu, Biochemistry 42(47): 13996-14003 (2003)). In this complex, the PNA/DNA/PNA triple helix and the PNA/DNA duplex both produce displacement of the pyrimidine-rich strand, creating an altered helical structure that has been shown to strongly provoke the nucleotide excision repair pathway and to activate the site for recombination with a donor DNA molecule (Rogers, et al., Proc Natl. Acad. Sci., USA, 99(26):16695-700 (2002)). A second tail-clamp PNA with a shorter Hoogsteen binding domain (tcPNA-684; FIG. 1C) was also tested. In vitro gel shift analysis shows a concentration dependent band shift when duplex DNA is incubated with increasing amounts (0, 0.2 μM, 0.4 μM, 0.8 μM and 1.2 μM) of PNA, confirming the formation of a triplex (Table 1). All three PNAs were found to bind to their specific CCR5 target sites in a plasmid substrate at physiological pH, however, tcPNA-679 showed superior target site binding.

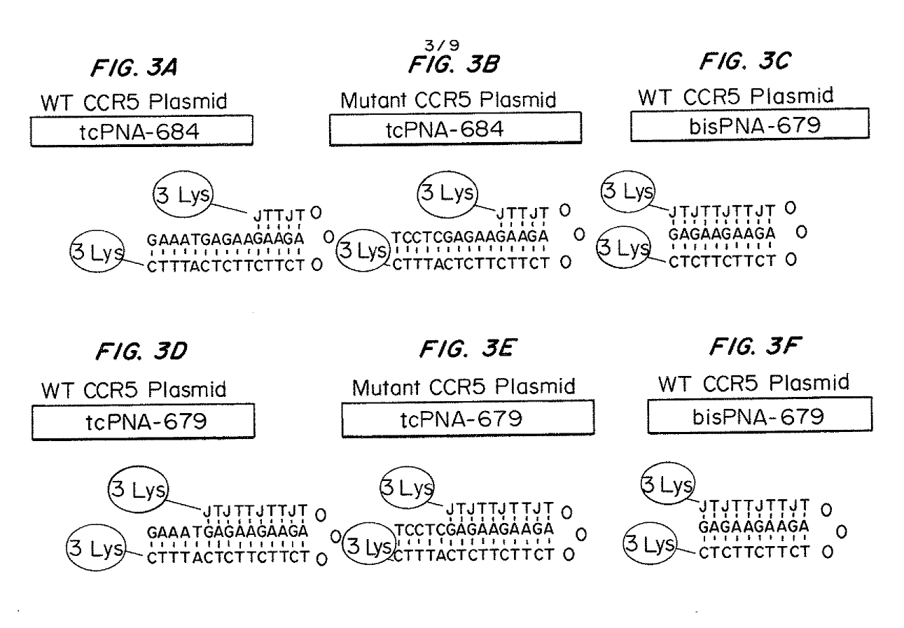

Next, the ability of the three PNAs to bind a CCR5 locus with mutations in the tail-binding sequence was tested using a mutant CCR5 plasmid. Mutations in the tail binding sequence greatly reduced the affinity of tcPNA-679 for its target (Table 1 and FIG. 3).

TABLE 1
Gel Mobility Shift Assay results: Percent unbound target duplex DNA (wildtype
or mutant CCR5) in the presence of increasing amounts of PNA (μM)

| | WT CCR5 Plasmid | | | | | Mutant CCR5 Plasmid | | | | |
|---|---|---|---|---|---|---|---|---|---|---|
| PNA [μM] | 0 | 0.2 | 0.4 | 0.8 | 1.2 | 0 | 0.2 | 0.4 | 0.8 | 1.2 |
| tcPNA-684 | 100 | 45 | 32.9 | 19.5 | 15.2 | 100 | 45 | 34.6 | 27.6 | 21.9 |
| tcPNA-679 | 100 | 17.9 | 5.3 | 3.7 | 2.6 | 100 | 52.9 | 20.9 | 8.6 | 7.9 |
| bisPNA-679 | 100 | 26.2 | 10.6 | 2.5 | 3 | — | — | — | — | — |

### Example 2

### Targeted Modification of the CCR5 Gene and Quantification in Human Cells

Materials and Methods

Cell Culture

THP-1 cells were maintained in RPMI supplemented with 10% FBS and L-Glutamine (GIBCO, Invitrogen, Carlsbad, Calif.). Human CD34⁺ stem cells were isolated from apheresis of granulocyte colony stimulating-factor (G-CSF) mobilized peripheral blood from healthy donors and then selected for using a Baxter 300i Isolex Device and cryopreserved (Yale Center of Excellence in Molecular Hematology, Yale University). Cells were thawed and maintained in StemSpan Serum-Free Expansion Media® supplemented with StemSpan™ CC110 cytokine mixture (100 ng/mL rh Flt-3 Ligand, 100 ng/mL rh Stem Cell Factor, 20 ng/mL rh IL-3, 20 ng/mL rh IL-6, StemCell Technologies Inc., Vancouver, BC Canada). THP-1 differentiation was induced by treatment with phorbol 12-myristate 13-acetate (PMA) (Sigma, St. Louis, Mo.) at a concentration of 50 ng/mL.

Electroporation of Molecules

THP-1 cells were electroporated at 350V, 12 ms, 1 pulse using a BTX Electo Square Porator ECM 830 (Harvard Apparatus, Inc., Holliston, Mass.) in 100 μL PBS with indicated concentrations of molecules. Following electroporation, cells were transferred into plates containing RPMI supplemented with 10% FBS and L-Glutamine. For analysis, cells were collected by centrifugation at the indicated times for genomic DNA or RNA isolation. Human CD34⁺ stem cells were thawed in media 24 hrs prior to transfection. Nucleofection was preformed using an Amaxa Human CD34 Cell Nucleofector® Kit according to the manufacturer's protocol (Lonza Group Ltd., Basel, Switzerland). Cells were then plated in media; and 24 or 48 hrs post-nucleofection, cells were collected by centrifugation for genomic DNA or RNA isolation.

Genomic DNA/RNA Isolation

Genomic DNA was isolated from tissue culture samples using the Wizard SV Genomic DNA Purification System (Promega, Madison, Wis.). DNA was eluted with 100 μL of dH₂O and diluted to 45 ng/μL for AS-PCR. Genomic DNA was isolated from THP-1 cells in 96-well plates using the Wizard SV 96 Genomic DNA Purification System (Promega, Madison, Wis.). THP-1 cells were induced with PMA for 48 hrs before genomic DNA was isolated. RNA was isolated using the Absolutely RNA Miniprep Kit (Stratagene, La Jolla, Calif.). Synthesis of cDNA from total RNA samples was performed using the SuperScript First-Strand Synthesis System (Invitrogen, Carlsbad, Calif.).

Allele-Specific PCR

Primers were designed to amplify a 400-bp region in CCR5. The allele-specific forward primer was designed to contain the specific 6-bp mutations at the 3′ end while the wild-type forward primer contained the wild-type CCR5 sequence at the same position. Primers were synthesized by the W. M. Keck Facility at Yale University (New Haven, Conn.) at a 40 nM scale and were diluted to a 10 μM stock in dH₂O. PCR products were separated on a 1% agarose gel and visualized using a gel imager. Wild-type forward primers paired with the universal reverse primer were used as a loading control. 3.2 μM donor 597 was added to an aliquot of untreated THP-1 genomic DNA with or without 6 μM tcPNA-579. Allele-specific (AS-PCR) was performed using untreated genomic DNA with or without PNA and donor to test the ability of the donor DNA to act as a primer or a template in the reaction. Forward primer, ASFUP: 5′ GTCCTGCCGCTGCTCTGAGG 3′(SEQ ID NO: 18), Reverse primer, ASR1: 5′ GTGTAAACTGAGCTTGCTCGCTC 3′ (SEQ ID NO: 19).

Results

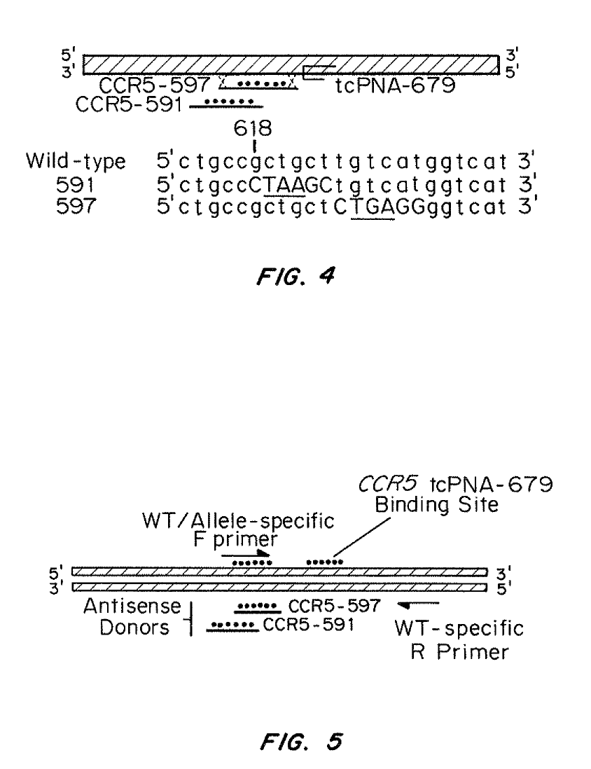

In the strategy for PNA-induced recombination (FIG. 4), PNA binding is expected to stimulate recombination between the chromosomal CCR5 gene and a co-transfected donor DNA. For this, two single-stranded antisense-oriented 60-nt donor DNAs, 591 and 597, were designed to be homologous to a portion of the CCR5 gene except for a central 6-bp segment intended to introduce, via recombination, an in-frame stop codon (FIG. 4) and to create a sufficient sequence change to be easily detectable by an allele-specific PCR (AS-PCR) assay (FIG. 5).

THP-1 cells, a human acute monocytic leukemia cell line that expresses CCR5 and can be infected with HIV-1, were used as an initial model to assay for targeted modification of CCR5 (Konopka and Duzgunes, AIDS Res Hum Retroviruses 18:123 (2002)). THP-1 cells were either mock transfected, transfected with donor 597 alone, or with donor 597 and one of the three PNAs. Donor concentrations were kept between 2-4 μM and PNA concentrations were fixed at 4 μM. After 48 hrs, genomic DNA was prepared and analyzed by AS-PCR (FIG. 5). Introduction of the targeted mutation into the CCR5 gene was produced at a low level by the donor DNA alone but was substantially induced by co-transfection of either PNA-679 or tcPNA-679 with the donor molecule while tcPNA-684 was less effective. As a further control, genomic DNA from untreated cells was spiked with donor DNA, and then the AS-PCR assay was performed. No signal was observed under these conditions, thus eliminating the possibility of false-positive results arising from persistence of the donor DNAs.

To better quantify the PNA-induced recombination and to establish the generation of long term, heritable genomic modification, cells were treated with either PNA-679 or tcPNA-679 (in both cases plus donor DNA) and then were diluted out into multi-well dishes. Single-cell clones were obtained and individually assayed for CCR5 gene modification.

TABLE 2
Quantification of PNA-induced CCR5 gene targeting

| | | Number of | |
|---|---|---|---|
| | | Clones with | |
| | Number of | Targeted | Targeting |
| PNA | Cells Assayed | Modification | Frequency |
| PNA-679 | 3680 | 20 | 0.5496 |
| tcPNA-679 | 1870 | 46 | 2.4696 |

Table 2 shows the quantification of PNA-induced CCR5 gene targeting by enumeration of CCR5-modified single-cell clones. PNA-679 gave a targeted modification frequency of 0.54%, consistent with the frequencies of induced recombination by PNAs of this design previously reported for the beta-globin gene target (Chin et al., Proc. Natl. Acad. Sci. USA, 105:13514-9 (2008)). However, tcPNA-679, which showed superior target site binding in vitro, yielded 46 positive clones out of 1870 cell clones, for an overall frequency of 2.46%. These results indicate that the tail-clamp PNA design improves the ability of triplex-forming PNAs to induce recombination. It is believed that this increased activity is due to increased binding affinity and to greater disruption of the underlying duplex target, thereby better provoking DNA repair and recombination.

It is believed that a targeted modification frequency of 2.46% is sufficient for therapeutic application because CCR5-deficient cells are known to have a selective advantage in the face of HIV infection. The addition of chloroquine as a lysosomal disrupting agent to increase PNA bioavailability; SAHA (suberoylanilide hydroxamic acid) as a histone deacetylase inhibitor to improve chromosome accessibility; and/or encapsulation of PNAs and oligonucleotides in PLGA nanoparticles are believed to further improve targeted modification frequency.

### Example 3

### Persistence of PNA-Induced Modification of CCR5

Materials and Methods

Allele-Specific Reverse Transcriptase PCR

Primers were designed to amplify a 667-bp region in CCR5. The allele-specific reverse primer was designed to contain the specific 6-bp mutation at the 3′ end while the wild-type reverse primer contained the wild-type CCR5 sequence. Forward primers were designed to bind in exon 2 with the reverse primer binding within exon 3, allowing for specific identification of cDNA as opposed to genomic DNA amplified products. The PCR products were separated on a 1% agarose gel and visualized using a gel imager. The forward primer paired with the wild-type reverse primer was used as a loading control.

Results

Reproducible CCR5 gene targeting was seen not only in THP-1 cells but also in another human cell line, K562, as determined by allele-specific PCR of 597 mutation in genomic DNA isolated from both cell types. Treated cells were also analyzed at the mRNA level using allele-specific reverse transcriptase PCR (AS-RT-PCR), confirming that the targeted modification is present in the mRNA expressed from the CCR5 gene. In addition, samples were taken intermittently from serially passaged cells that had been treated with PNA-679 and donor 597. AS-PCR of these cell populations confirmed that cells containing the targeted 597 mutation persisted for at least 98 days in culture, establishing that a heritable sequence change had been generated, and that the modified cells are viable, further eliminating concern regarding any potential PCR artifacts that could arise from transient persistence of the donor DNA. Similarly, five clones were maintained in culture by serial passage for thirteen months, and all five were confirmed to retain the targeted modification in CCR5.

### Example 4

### Isolation and Characterization of Single Cell Modified Clones and its Effect on Protein Expression and HIV-1 Infectibility

Materials and Methods

Infectibility

THP-1 cells were induced to differentiate into adherent macrophage-like cells by treatment with PMA. Cells were plated at 2×10⁵ cells/well in 96-well plates and treated with 50 ng/mL PMA for 48 hrs at 37° C. and washed thoroughly. Seven days after differentiation, PMA-treated THP-1 (THP-1/PMA) cells were exposed to HIV-1_{BaL} at an MOI of 1. After a 4 hr incubation at 37° C. with the virus, cells were washed three times and cultured in 10% RPMI. Infection was monitored by the viral p24 level in harvested culture supernatants (harvested on days 4, 6, 8, 10 and 12 post-infection), using enzyme-linked immunosorbent assay (ELISA) plates obtained from PerkinElmer Life Sciences (Waltham, Mass.). The results are expressed as the mean and standard deviation (SD) of duplicate determinations from two wells. To calculate moi, HIV_{BaL} was titrated on 1×10⁵ primary human monocyte derived macrophages seeded in 96 well plates and intracellular p24 antigen was measured by flow cytometry. Based on this 100 ng p24 gag antigen equivalents per well was determined to be the TCID₅₀ (moi=0.5).

Quantitative Allele-Specific Real-Time PCR Quantitative PCR was performed using Brilliant SYBR Green qPCR reagents on the Mx3000p real-time PCR system (Stratagene). Fluorescence intensity was monitored in real-time, and cycle threshold cycles (CTs) were calculated based on dRn fluorescence with an adaptive baseline using the software supplied with the MX3000p. Comparative quantification was performed by comparing the CTs obtained from amplification of CCR5 to those observed using gene specific primers (forward: 5′ ACCTTTGGGGTGGTGACAAGTGTG 3′ (SEQ ID NO: 20) and reverse: 5′ TCTCCCCGACAAAGGCATAGATGA 3′ (SEQ ID NO: 21) as a normalizer. Relative mutant CCR5 abundance was calculated using the −ΔΔCT method (Stratagene). All assays were preformed in duplicate.

Taqman Assay for RNA Induction

To test CCR5 mRNA expression, cDNA was prepared from total RNA samples from PMA-induced and -uninduced THP-1 cells. cDNA was used in PCR reactions containing Taqman Universal master mix, premixed Taqman probes, and reference dye (Applied Biosystems, Foster City, Calif.) in a final volume of 25 μL. Data was analyzed as previously described (Bindra and Glazer, Cancer Lett, 252(1):93-103 (2007), Bindra et al., Cancer Res, 65(24):11597-604, (2005)). The amplification scheme was: 50° C. for 2 mins, 95° C. for 10 mins, 95° C. for 15 s and 60° C. for 1 min for 45 cycles preformed using the Mx3000p real-time PCR system (Stratagene). Fluorescence intensity was monitored in real-time, and cycle threshold cycles (CTs) were calculated based on dRn fluorescence with an adaptive baseline using the software supplied with the MX3000p. Comparative quantification was performed by comparing the CTs obtained from amplification of CCR5 to those observed for 18S rRNA as a normalizer. Relative mRNA abundance was calculated using the −ΔΔCT method (Stratagene). CCR5 probes were FAM-labeled, and the 18S probe was MGB-labeled (obtained from Applied Biosystems). All assays were preformed in duplicate.

### Cell Surface Staining

To test for the presence of CCR5 at the cell surface, 2×10⁶ cells were incubated with PMA dissolved in DMSO to a final concentration of 50 ng/mL in culture medium, or mock treated with DMSO alone for 48 hrs. Cells were then collected, washed with PBS, and then washed with CCR5 staining buffer (2% FBS certified (GIBCO), 1% Sodium Azide (10% w/v solution), and PBS (GIBCO)). Cell pellets were then resuspended in CCR5 staining buffer and Function Grade Purified anti-mouse CD16/32 (eBioscience, Inc., San Diego, Calif.) was added to THP-1 cells for blocking Cells were allowed to incubate at room temperature for 10 minutes before being split into fresh tubes containing either a PE-conjugated anti-hCCR5 or Mouse IgG₁ Isotype Control antibody (R&D Systems, Inc., Minneapolis, Minn.) and kept on ice for 20 minutes. Cells were then pelleted, and the supernatant was removed. 500 μL of CCR5 staining buffer was used to wash the cells before they were resuspended in PBS for FACS. Samples were analyzed on a FACSCalibur with CellQuest software (Becton Dickinson, San Jose, Calif.)

Results

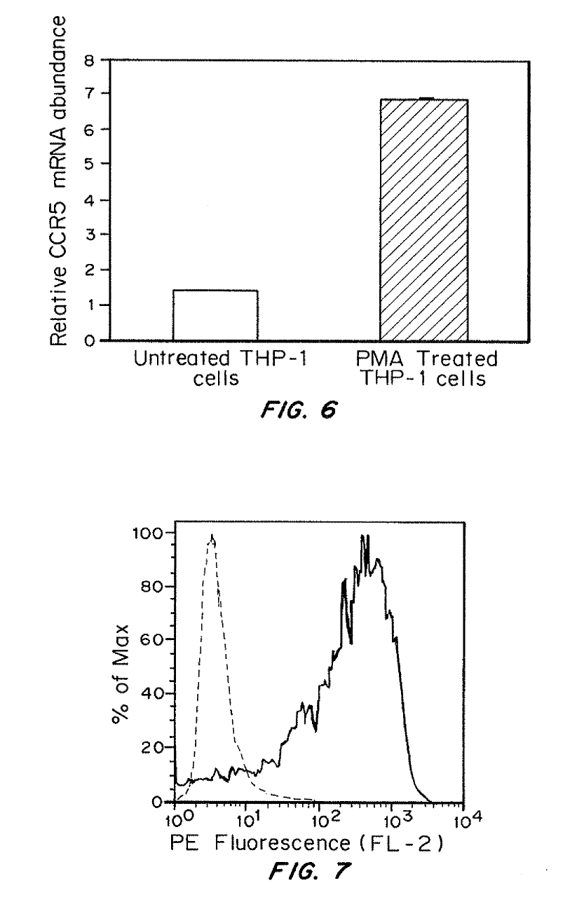

To generate a cell population with both CCR5 alleles modified, one clone carrying the 597 modification (confirmed by allele-specific PCR and also by genomic DNA sequencing to have the modified allele) was expanded and transfected with tcPNA-679 and DNA donor 591. From this treated population, a doubly mutant clones with both alleles modified were identified by AS-PCR. The frequency that such clones were induced from heterozygous cells was 0.98% (18/1840). The parental THP-1 cells, the heterozygous mutant (Mut⁵⁹⁷/WT) clone, and a double-mutant (Mut⁵⁹⁷/Mut⁵⁹¹) clone were tested for targeted CCR5 modification at the protein level. For this, the ability of phorbol myristate acetate (PMA) to promote differentiation of THP-1 cells into a macrophage-like state was utilized, thereby yielding high levels of CCR5 cell surface expression (Konopka and Duzgunes, AIDS Res Hum Retroviruses, 18(2):123-31 (2002), Jagodzinski, Viral Immunol, 12:23 (1999)). In the parental THP-1 cells, PMA treatment increased CCR5 mRNA expression approximately 5-fold, as assessed by a Taqman real-time PCR assay, and increased CCR5 protein cell surface expression 17-fold as determined by antibody staining and flow cytometry (FIGS. 6 and 7). The Mut⁵⁹⁷/WT heterozygous clone was treated with PMA and determined by flow cytometry to have an approximately 50% reduction in cell surface expression of CCR5 compared to WT/WT cells. The double-mutant clone, Mut⁵⁹⁷/Mut⁵⁹¹, had minimal cell surface staining for CCR5, essentially at the background level of the isotype staining in the assay, even after PMA treatment. These results, quantified in FIG. 8, show at the protein level that PNA plus donor DNA treatment of THP-1 cells produces functional disruption of the CCR5 gene. This is consistent with the introduction of stop codons by donors 591 and 597 to yield truncated proteins that fail to properly localize to the cell membrane, thereby mimicking the effect of the naturally occurring CCR5-delta32 mutation.

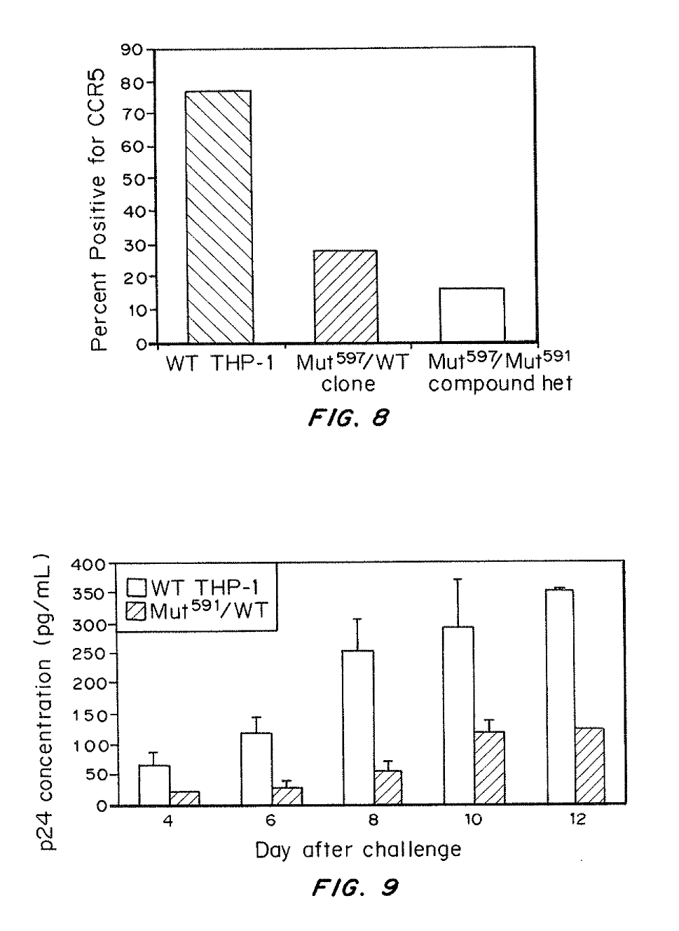

Since the goal of CCR5 gene targeting is to create a cell population that is resistant to HIV-1 entry, the infectability of CCR5-modified THP-1 cells with R5-tropic HIV-1 infection was tested. Following a single treatment of THP-1 cells with tcPNA-679 and DNA donor 591, as above, Mut⁵⁹¹/WT heterozygous clones were isolated. One randomly selected clone was expanded, and was seeded into multi-well plates at 2×10⁵ cells per well in multiple replicates. In parallel, parental THP-1 cells, wild-type at the CCR5 locus, were seeded in the same manner. The cells were treated with PMA for 48 hrs to induce CCR5 cell surface expression and then were challenged by addition of live HIV-1_{BaL} at an MOI of 1. At 4, 6, 9, 10, and 12 days following infection, supernatants were harvested and frozen. Upon completion of the time course, the supernatants were thawed and analyzed by ELISA for core protein p24 antigen levels as a measure of viral infection of the cells. The ELISA results indicate a substantial decrease in HIV-1 infection of the cells modified at the CCR5 locus by treatment with tcPNA-679 and DNA donor 591 (FIG. 9), demonstrating functional knockout of the CCR5 co-receptor (correlating further with the CCR5 DNA, RNA, and protein expression analyses) and establishing the endpoint of reduced HIV-infectibility of PNA-modified human cells.

### Example 5

### Modification of Human HSCs and their Engraftment Capability

Materials and Methods

Mouse Transplantation

All animal use was in accordance with the guidelines of the Institutional Animal Care and Use Committee (IACUC) of the University of Massachusetts Medical School and The Jackson Laboratory and conformed to the recommendations in the Guide for the Care and Use of Laboratory Animals (Institute of Laboratory Animal Resources, National Research Council, National Academy of Sciences, 1996). Targeted or untreated human CD34⁺ cells were resuspended in RPMI supplemented with 1% fetal calf serum. NOD.Cg-Prkdc^{scid} Il2rg^{tm1Wjl}/Sz Tg(HLA-A2/H2-D/B2m)1Dvs/Sz (NOD-scid IL2ry^{null} HLA-A2.1) mice were obtained from the research colony maintained by L.D.S. at The Jackson Laboratory. 50 μL, of T cell depleted human umbilical cord blood cells containing 3×10⁴ CD34⁺ cells were injected into newborn NOD-scid IL2ry^{null} HLA A2.1 mice by intracardiac injection. 12 weeks post injection mice were bled and analyzed by FACS to determine engraftment of human hematopoietic cells. Mice were sacrificed, and various tissues were harvested and flash frozen. CD4-positive cells were isolated from one fresh spleen sample from a PNA plus donors-treated mouse using BD IMag Anti-Human CD4 Particles according to the manufacturer's protocol (BD Biosciences, San Jose, Calif.). Genomic DNA was isolated from tissues by phenol/chloroform extraction and analyzed by quantitative allele-specific real-time PCR.

Results

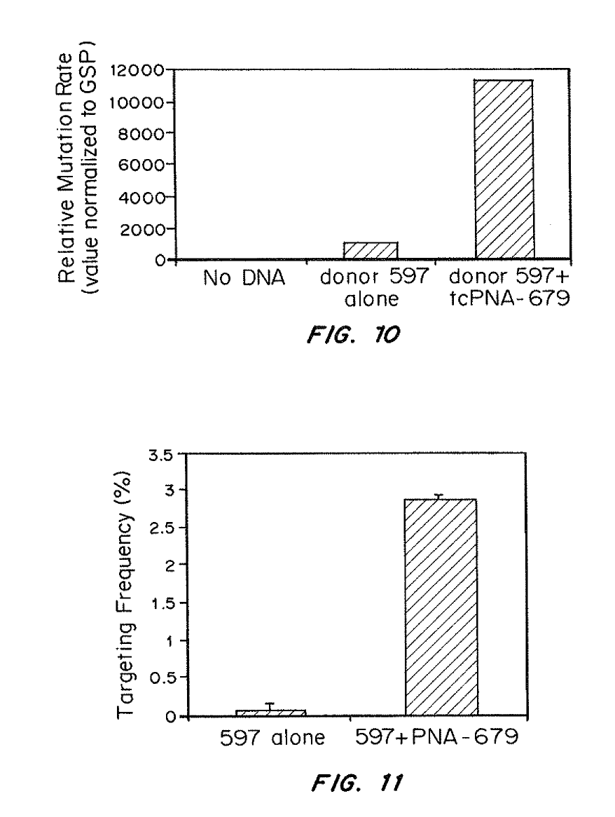

Ex vivo modification of hematopoietic stem cells (HSCs) from HIV-1-infected patients is a method to create a renewable source of virus-resistant immune cells. Importantly, human CD34 HSCs have been shown to remain uninfected even in HIV-1-infected patients (Shen et al., J Virol, 73:728 (1999)). Hence, modification of such cells would provide a source of HIV-1-resistant cell lineages. An optimized protocol for transfection of human CD34⁺ cells that not only yields high transfection efficiency but also maintains CD34 expression was used. The ability of tcPNA-679 plus donor DNA 597 to modify the CCR5 gene in primary human CD34⁺ cells was tested next. The cells were transfected with tcPNA-679 and donor 597, with donor 597 alone, or with buffer alone; and 24 or 48 hrs post-transfection cells were harvested for analysis of genomic DNA and mRNA. The 597 mutation was detected in the CD34⁺ cells treated with donor alone, but in the population transfected with tcPNA-679 and donor 597, the level of modification at the target site was substantially higher. The stimulation of gene modification by tcPNA-679 was determined to be approximately 12-fold as quantified by real-time AS-PCR (FIG. 10). To further quantify the targeting frequencies, a standard curve was established by mixing genomic DNA from CCR5 heterozygous and wild-type cells in defined ratios. Real-time AS-PCR was performed on the DNAs from the experimental samples, and the results were quantified based on the standard curve. CD34+ cells transfected with donor DNA alone were calculated to have a modification frequency of 0.03% whereas the PNA-treated CD34+ cells were estimated to have a modification frequency of 2.8%, a more than 90-fold increase due to the effect of the PNA (FIG. 11). Importantly, targeted modification in the CD34+ cells was also confirmed at the mRNA level by AS-RT-PCR (FIG. 4c), indicating that the successfully modified CD34+ HSCs express mutant CCR5 mRNA. These results support the use of PNA-induced genomic modification in human primary cells.

It is also important to note that the level of PNA-induced gene modification of CCR5 was comparable or even higher in CD34⁺ cells than in THP-1 cells. These results show not only that primary human HSCs are susceptible to targeted gene modification mediated by triplex-forming PNAs, but also that effective gene modification in primary HSCs is highly dependent on the stimulatory effect of the PNAs. While some studies have reported genome modification in human stem cells and various human and rodent cell lines using single stranded oligonucleotide donor DNAs alone (Pierce et al., Gene Therapy, 10(1):24-33 (2003) and Goncz, et al., Oligonucleotides, 16:213-24 (2006)), these results indicate that primary HSCs appear to be much less susceptible to this approach and indicate that the use of triplex-forming PNAs to stimulate recombination is needed.

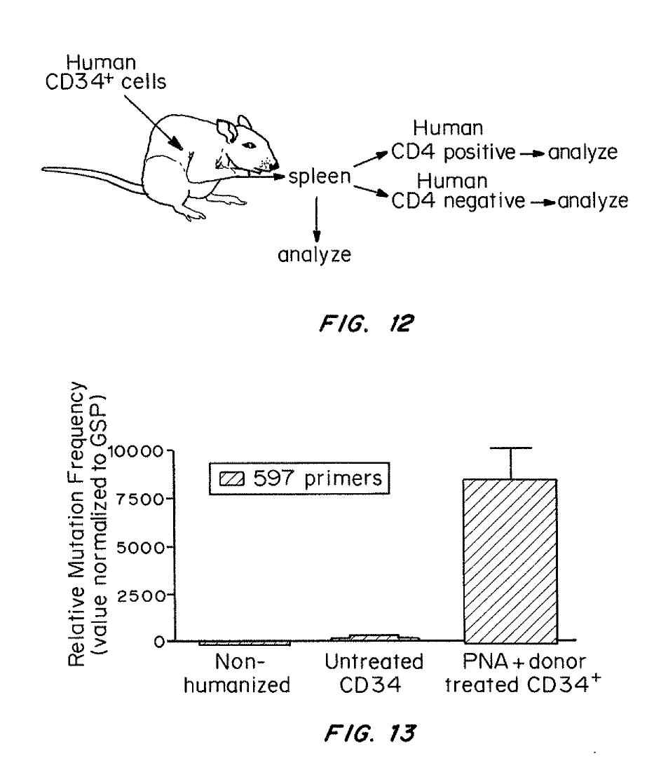

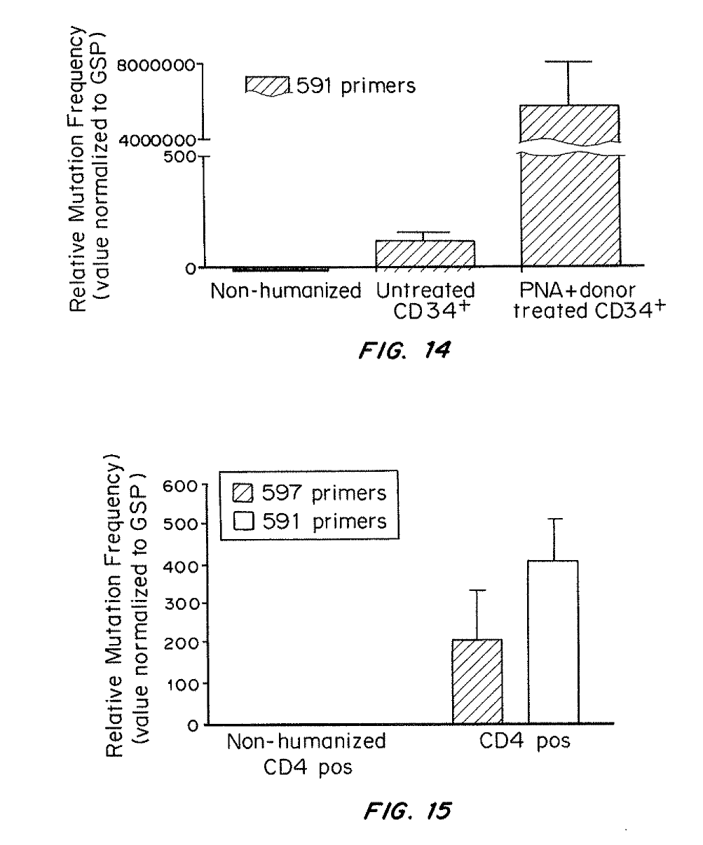

PNA-modified CD34⁺ HSCs were next tested for their ability to mediate human hematopoietic stem cell engraftment in immune deficient mice. CD34⁺ HSCs were transfected, as above, with tcPNA-679 plus both donor 591 and donor 597. Successful modification of the CCR5 gene was first confirmed by AS-PCR after 24 hrs in an aliquot of the treated cell population. The treated cells were then transplanted into newborn NOD-scid IL2ry^{null} LA A2.1 mice via intra-cardiac injection (FIG. 12). At four months post-transplant, peripheral blood cells from the engrafted mice were harvested and FACS was used to confirm engraftment of the human HSCs in the mice by the identification of cells carrying human cell surface markers, including CD3, CD4, CD8, CD20, and CD45. AS-PCR on genomic DNA isolated from the mouse spleen was then performed to test for the presence of the targeted modification of the human CCR5 gene in donor human cells in the engrafted mice. The results show the presence of the targeted 591 and 597 mutations in the human CCR5 gene persisting more than four months post-transplant in the spleen of the mice transplanted with PNA-targeted human CD34⁺ cells, but not in the spleens of control mice (FIGS. 13 and 14). Representative results from one of two mice is shown; the results from the other mouse were similar. In addition, CD4⁺ cells were isolated from the spleen at five months post-transplant, and these also showed the PNA-treated modification in CCR5 (FIG. 15). These data show that modification of primary HSCs via PNA and donor DNA transfection does not disrupt their engraftment capability and still allows for multi-lineage repopulation, establishing that this approach to targeted gene modification could provide the basis for a clinical therapy.

### Example 6

### Targeted Modification of the Δ32 Mutation Site in Cells Using Donor DELTA32JDC

Materials and Methods

Donor Oligonucleotides and Allele-Specific PCR Primers

Donor oligonucleotide: DELTA32JDC
(with phosphothioate linkages at first 3
and last 3 bases).
(SEQ ID NO: 15)
5′GATGACTATCTTTAATGTCTGGAAATTCTTCCAGAATTAATTAAGA
CTGTATGGAAAATGAGAGC3′
Allele-specific PCR forward primer:
(SEQ ID NO: 22)
JDCDELASF: 5′CATTTTCCATACAGTCTTAATT3′
Allele-specific PCR reverse primer:
(SEQ ID NO: 19)
ASR1: 5′GTGTAAACTGAGCTTGCTCGCTC 3′.

Allele-Specific PCR Conditions

1) 94° C. 2 minutes,

2) (94° C. 15 seconds, 50° C. 30 seconds, 68° C. 30 seconds)×40 cycles,

3) 68° C. 1 minutes.

Standard PCR reactions were assembled using Platinum Taq (Invitrogen) to provide a hot start and enhance specificity. Reaction products were run on a 1% agarose gel.

Results

DELTA32JDC was designed as a donor which spans the Δ32 mutation. This donor can be used to target the wildtype allele in cells heterozygous at the Δ32 locus. This approach would be particularly useful in treating patients that are heterozygous for the Δ32 mutation. The DELTA32JDC donor was designed to accommodate both allele-specific PCR (AS-PCR) and Restriction endonuclease selective PCR (REMS PCR) using the heat resistant endonuclease TsprI. REMS PCR was found to be ineffective at detecting small allele frequencies in total genomic DNA. Therefore AS-PCR was used to detect modification at the CCR5 locus. K562 cells were electroporated with DELTA32JDC donor and 2 uM or 4 uM tcPNA679. AS-PCR of total genomic DNA revealed a donor fragment dose-dependent modification of the target site.

### Example 7

### Targeted Modification of the Δ32 Mutation Site in Cells Using Donor DELTA32JDC2

Materials and Methods

Donor Oligonucleotide and Primer Sequences

Donor oligonucleotide:
DELTAJDC2 donor has the sequence:
(SEQ ID NO: 16)
5′CCCCAAGATGACTATCTTTAATGTCTGGAACGATCATCAGAATTGA
TACTGACTGTATGGAAAATG 3′
Allele-specific PCR forward primer:
(SEQ ID NO: 23)
JDCASF3: 5′CCATACAGTCAGTATCAATTCTGATGATCG3′
Allele-specific PCR reverse primer:
(SEQ ID NO: 19)
ASR1 5′GTGTAAACTGAGCTTGCTCGCTC3′

PCR Conditions

1) 94° C. 2 minutes,

2) (94° C. 15 seconds, 50° C. 30 seconds, 68° C. 30 seconds)×40 cycles,

3) 68° C. 1 minutes.

Standard PCR reactions were assembled using Platinum Taq (Invitrogen) to provide a hot start and enhance specificity.

Results

Donor DELTAJDC2 is a modified version of DELTAJDC, designed for detection by allele-specific PCR. DELTAJDC2 incorporates a 7 bp mutation which inserts two stop codons in tandem. THP-1 cells were electroporated with various concentrations of DELTAJDC2 donor and a fixed 4 uM concentration of tcPNA679, and analyzed by allele-specific PCR of total genomic DNA. Allele-specific PCR indicated that increasing concentrations of donor oligonucleotide (0, 2 μM, 4 μM, 6 μM, 8 μM, 10 μM yielded higher frequencies of modification at the target site in dose-dependent manner. Additionally, THP-1 and K562 cells were electroporated with 4 uM DELTAJDC2 donor with or without 4 uM concentration of tcPNA679. Allele-specific-PCR indicated that tcPNA679 provided an increase in gene targeting over DELTAJDC2 alone in both cell types.

### Example 8

### Targeted Modification of the Δ32 Mutation Site in Cells Using Donor DELTA32RSB

Material and Methods

Donor Oligonucleotide and Restriction Endonuclease Selective PCR

Donor Oligonucleotide:
DELTA32RSB (with phosphothioate linkages at
first 3 and last 3 bases):
SEQ ID NO: 17)
(5′GATGACTATCTTTAATGTCTGGAAATTCTACTAGAATTGATACTGA
CTGTATGGAAAATGAGAGC3′
Restriction endonuclease selective PCR forward
primer:
(SEQ ID NO: 24)
RSB32PSPF: 5′GCTCTCATTTTCCATACAGTCAGTATCAATCC3′
Restriction endonuclease selective PCR reverse
primer:
(SEQ ID NO: 25)
CCR5RSBR2: 5′GTAGGGAGCCCAGAAGAGAAAATAAACAATCAT3′

Restriction Endonuclease Selective PCR (REMS-PCR) Conditions

1) 94° C. 2 minutes,

2) (94° C. 15 seconds, 68° C. 1 minute)×40 cycles,

3) 68° C. 1 minutes.

One hundred ng of genomic DNA were used in a standard PCR reaction using Taq polymerase (Invitrogen). Ten units (U) of PspGI were used in the REMS-PCR reactions. PCR reactions were run on a 1% agarose gel.

Results

Like DELTA32JDC, Donor DELTA32RSB was designed as a donor which spans the Δ32 mutation. This donor can be used to target the wildtype allele in cells heterozygous at the Δ32 locus. This approach would be particularly useful in treating patients that are heterozygous for the Δ32 mutation. Unlike DELTA32JDC, DELTA32RSB was designed to be detected by restriction endonuclease selective PCR (REMS-PCR) using the thermostable endonuclease PspGI. REMS-PCR works in the following manner. PCR is conducted in the presence or absence of PspGI. The forward primer included in the PCR reaction introduces a base pair change to create the PspGI restriction consensus such that amplification could not occur in the presence of PspGI. Without PspGI amplification does indeed occur as the PCR product is not under constant assault by the enzyme and is not degraded. The forward primer is designed such that it does not introduce a PspGI site when amplifying off of a DNA template containing the mutation incurred by DELTA32RSB. Thus these products are not subject to degradation in the presence of PspGI and are preferentially amplified over WT product, which are degraded by PspGI. K562 cells were electroporated with 2 uM DELTA32RSB donor and 4 uM tcPNA679 Following REMS-PCR of total genomic DNA, PCR product was detected for both mock and cells transfected with DELTA32RSB donor in the absence of PspGI. However, in the presence of PspGI, PCR product was only detected cells transfected with DELTA32RSB donor, indicating modification of the target site in cells. This result also indicates that REMS-PCR is a viable method for confirming modification has occurred at the target site.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. A recombinagenic or mutagenic composition between six and fifty peptide nucleotides in length comprising
two single-stranded molecules having sequences which can bind or hybridize to a target duplex nucleic acid molecule comprising sixteen base pairs and including a polypurine:polypyrimidine stretch inducing strand invasion and displacement to form a triplex with the two single-stranded molecules,
wherein the first single-stranded molecule contains a portion of nine or more nucleobases in length that binds to the target duplex by Watson-Crick binding and a tail sequence of up to fifteen nucleobases that binds to the target duplex outside of the triplex, and
wherein the second single stranded molecule comprises six or more nucleobases in length and binds to the target duplex by Hoogsteen binding
wherein the target duplex is a human gene in a living cell that encodes a cell surface receptor for HIV.

2. The composition of claim 1 wherein the Watson-Crick binding portion is between about 9 and 30 nucleobases in length, including a tail sequence of up to 15 nucleobases.

3. The composition of claim 2 wherein the Watson-Crick binding portion is between about 10 and 25 nucleobases in length, including a tail sequence of up to 10 nucleobases.

4. The composition of claim 3 wherein the Watson-Crick binding portion is between 15 and 25 nucleobases in length, including a tail sequence of 5 to 10 nucleobases.

5. The composition of claim 1 wherein the Hoogsteen binding portion is between about 6 and 15 nucleobases, inclusive.

6. The composition of claim 1 wherein the base composition of the triplex-forming molecules may be homopyrimidine.

7. The composition of claim 1 wherein the two single-stranded molecules are peptide nucleic acids comprising at least 8 pyrimidines.

8. The composition of claim 1 wherein the two single-stranded molecules are connected by a linker to form one molecule.

9. The composition of claim 1 further comprising one or more donor oligonucleotides between 4 and 100 nucleotides in length, more preferably between 25 and 80 nucleobases.

10. The composition of claim 9 wherein the donor fragment is linked to the triplex forming composition.

11. The composition of claim 10 wherein the donor fragment is between 1 to 800 nucleotide bases, more preferably 25 to 74 nucleotide bases, most preferably about 50 nucleotide bases, from the target binding site of the triplex-forming molecule.

12. The composition of claim 1 wherein the human gene is a chemokine gene selected from the group consisting of CXCR4, CCR5, CCR2b, CCR3, and CCR1.

13. The composition of claim 12 wherein the sequence of the two single-stranded molecules bind to a target sequence in or near the polypurine stretch between nucleotide 679 and 690 of the human CCR5 gene.

14. The composition of claim 13 wherein recombination of the donor sequence at the target site induces a change the target duplex nucleic acid that mimics the Δ32 mutation.

15. The composition of claim 9 wherein the donor oligonucleotide comprises one or more nucleotide mutations, deletions or insertions relative to the target duplex DNA nucleotide sequence.

16. The composition of claim 15 wherein the mutation, deletion or insertion results in a deficiency in a cell surface receptor encoded by the human CCR5 gene selected from the group consisting of reduced expression of the receptor, defects in transport of the receptor to the cell surface, reduced stability of the receptor protein, reduced binding of HIV by the receptor and defects in endocytosis of the receptor.

17. The composition of claim 1 comprising a tail clamp peptide nucleic acid with the sequence from N-terminus to C-terminus-Lys-Lys-Lys-JTJTTJTTJT-OOO-TCTTCTTCTCATTTC-Lys-Lys-Lys (SEQ ID NO: 5), where J=pseudoisocytosine and o=flexible 8-amino-3,6-dioxaoctanoic acid, 6-aminohexanoic acid monomers.

18. The composition of claim 1 wherein the donor oligonucleotide comprises one or more point mutations that cause missense or nonsense mutations in the target duplex DNA nucleotide sequence wherein the missense or nonsense mutations result in a frameshift or deletion in the target duplex DNA.

19. The composition of claim 1 comprising one or more donor oligonucleotides having a sequence selected from the group consisting of 5′ AT TCC CGA GTA GCA GAT GAC CAT GAC AGC TTA GGG CAG GAC CAG CCC CAA GAT GAC TAT C 3′ (SEQ ID NO: 13) and 5′ TT TAG GAT TCC CGA GTA GCA GAT GAC CCC TCA GAG CAG CGG CAG GAC CAG CCC CAA GAT G 3′ (SEQ ID NO: 14).

20. The composition of claim 1 wherein the donor oligonucleotide spans the Δ32 mutation.

21. The composition of claim 1 wherein the donor oligonucleotide is selected from the group consisting of 5′GATGACTATCTTTAATGTCTGGAAATTCTTCCAGAATTAATTAAGA CTGTATGGAAAATGAGAGC3′ (SEQ ID NO: 15), 5′CCCCAAGATGACTATCTTTAATGTCTGGAACGATCATCAGAATTGA TACTGACTGTATGGAAAATG 3′ (SEQ ID NO: 16), and
5′GATGACTATCTTTAATGTCTGGAAATTCTACTAGAATTGATA CTGACTGTATGGAAAATGAGAGC3′ (SEQ ID NO: 17).

22. A method for targeted recombination or mutation of a gene encoding a cell surface receptor for HIV comprising contacting living cells with the composition of claim 1.

23. A method for prophylaxis or treatment of HIV infection in subjects with or at risk of developing an HIV infection comprising
a) isolating cells from a host,
b) contacting the cells ex vivo with the composition of claim 1,
c) expanding the cells in culture, and
d) administering the cells to a subject in need thereof.

24. The method of claim 23 wherein the cells are resistant to infection by one or more strains of HIV.

25. The method of claim 24 wherein the cells are resistant to R5-trophic HIV strains.

26. The method of claim 23 wherein the cells are isolated from the subject to be treated or a syngenic host.

27. The method of claim 23 wherein the cells are CD34⁺ cells.

28. The method of claim 23 further comprising differentiating the cells into CD4⁺ cells prior to step d).

29. A method for prophylaxis or treatment of HIV infection in subjects with or at risk of developing an HIV infection comprising administering to a subject in need thereof the composition of claim 1.

30. A cell line generated by the method of claim 22.
